(19) United States
(12) Patent Application Publication
(10) Pub. No.: US 2014/0303037 A1
(43) Pub. Date: Oct. 9, 2014

(54) PATTERNING METHOD

(71) Applicants: Robert D. Short, Burnside (AU); Endre J. Szili, Athelstone (AU); Sameer Al-Bataineh, Mawson Lakes (AU)

(72) Inventors: Robert D. Short, Burnside (AU); Endre J. Szili, Athelstone (AU); Sameer Al-Bataineh, Mawson Lakes (AU)

(73) Assignee: UNIVERSITY OF SOUTH AUSTRALIA, Adelaide, SA (AU)

(21) Appl. No.: 14/342,235

(22) PCT Filed: Aug. 31, 2012
(86) PCT No.: PCT/AU2012/001038

§ 371 Date: Jun. 24, 2014

(30) Foreign Application Priority Data
Sep. 1, 2011 (AU) 2011903525
Sep. 20, 2011 (AU) 2011903860

(51) Int. Cl. B01J 19/00; C12N 5/00; G01N 33/543; H01J 37/32

(52) U.S. Cl. CPC B01J 19/0046; H01J 37/32366; C12N 5/0068; G01N 33/54353
USPC 506/14; 427/535; 435/402; 506/32; 506/26; 506/16; 506/18; 506/19; 428/195.1

(57) ABSTRACT

A method of patterning a surface of a substrate comprising: (a) applying a coating to the surface to form a coated surface, and (b) treating the coated surface with a patterned microplasma comprising a plurality of localised microplasma discharges such that localised regions of the coated surface are selectively exposed to the localised microplasma discharges to form exposed localised regions and unexposed regions that have not been substantially exposed to a microplasma discharge; wherein the coating at the exposed localised regions is modified by the patterned microplasma and the coating at the unexposed regions is substantially unmodified to form a patterned surface on the substrate.

### PRIORITY DOCUMENTS

The present application claims priority from Australian Provisional Patent Application No. 2011903525 entitled “PATTERNING METHOD” filed on 1 Sep. 2011 and Australian Provisional Patent Application No. 2011903860 entitled “PATTERNING METHOD II” filed on 20 Sep. 2011 each of whose contents are hereby incorporated by reference in their entirety.

### TECHNICAL FIELD

The present invention relates to a method of patterning a substrate such as a microfluidic lab-on-a-chip device, a biosensor, an implantable “biomaterial”, a tissue engineering scaffold or support, a cell expansion surface or cell array.

### BACKGROUND

Spatially controlled surface modification is important for the development of microfluidic lab-on-a-chip devices, biosensors and other diagnostics tools, implantable “biomaterials”, tissue engineering scaffolds and supports, cell culture and cell expansion surfaces for cell-based therapies. Techniques associated with the controlled positioning of target molecules on a substrate are known as “patterning” of these molecules. The control of the position or distribution of target molecules on a substrate is useful for a number of scientific and technological applications. For instance, surface-bound biological molecules can be used as multiplex surface-capture assays providing hundreds to thousands of data points per experiment; DNA polynucleotides can be patterned onto a glass substrate to produce DNA microarray chips; RNA polynucleotides can be patterned onto a substrate to produce RNA microarray chips; proteins and/or peptides can be patterned onto a substrate to produce protein/peptide arrays; and sugars can be patterned onto a substrate to produce sugar arrays. Furthermore, the spatial organisation of particular molecules is thought to influence structure, function and replication of cells both in vivo and when cultured in vitro. This latter technique may be particularly useful for the in vitro culture of cells that are difficult to maintain or expand in vitro in a desired form. For example, cell differentiation may be controlled by confining cells spatially on a surface and restricting spread of the cells using suitable surface treatments.

A number of techniques have been used to pattern substrate surfaces. In self-assembled patterning, the physical and/or chemical properties of a molecule or combination of molecules are exploited under specific conditions to produce distributions of molecules with known non-random spatial properties. In directed lithographic patterning, the position of the molecules is externally controlled using a physical component such as a patterned mask, stamp, mould, stencil, template or the like, which is contacted to the substrate to mediate transfer of the pattern to a substrate. Directed writing patterning uses a serial approach to transfer a pattern, often from a computer-based representation such as a computer assisted design (CAD) drawing, to a substrate. However, for patterning of biological molecules in particular, these techniques can potentially cause undesirable denaturation, aggregation and conformational changes of the biomolecule. Non-uniform drying of printed spots and subsequent blocking of the entire substrate surface can present further complications.

Plasmas, which are electrically-excited ionized gases, can be utilised to pattern a substrate surface. Upon excitation, a non-equilibrium present between high-temperature electrons and the remaining plasma components enables their use in the physicochemical processing of a wide variety of materials. For example, they can modify pre-existing surfaces or deposit thin films without altering the properties of the underlying bulk material (France et al. 1997; Ward et al. 1993; Ward et al. 1995). Such a method is disclosed in United States Patent Application Publication No. 2008/0220516 which discloses the use of a physical mask insert in contact with the surface of the substrate. However, the commercial use of plasma patterning has been limited by the need to use physical masks in contact with the surface of the substrate, or by the use of chemical etchants and solvents. In these cases, the patterning procedure is a multistep process.

Microplasmas, operated at or near atmospheric pressure, are electrically-driven, low temperature and non-equilibrium plasmas that are geometrically confined to small dimensions (micrometers to millimetres). Microplasmas create highly reactive environments comprising ions, excited species, radicals, and photons. Microplasma devices have been developed for localized surface modification using a method referred to as “plasma printing” (Klages et al. 2007; Kreitz et al., 2005). However, these methods are also undesirable due to the requirement for intimate contact between the substrate and a plasma stamp or mask.

United States Patent Application Publication No 2011/0136162 discloses an alternative patterning method that involves moving a microplasma nozzle relative to a surface of a substrate in a predetermined pattern to create a pattern on the surface. Whilst this method does not require the specific use of a mask it is still relatively inefficient because it involves formation of individual microplasma treated areas sequentially.

There is a need for substrate patterning methods that overcome one or more problems associated with prior art patterning methods and/or that provide an alternative to prior art patterning methods.

### SUMMARY

The present invention arises from research into plasma patterning of substrate surfaces and, in particular, our finding that patterned substrate surfaces can be formed using patterned microplasma without the need for a mask on the substrate.

In a first aspect, the present invention provides a method of patterning a surface of a substrate comprising:
(a) applying a coating to the surface to form a coated surface, and
(b) treating the coated surface with a patterned microplasma comprising a plurality of localised microplasma discharges such that localised regions of the coated surface are selectively exposed to the localised microplasma discharges to form exposed localised regions and unexposed regions that have not been substantially exposed to a microplasma discharge;
   wherein the coating at the exposed localised regions is modified by the patterned microplasma and the coating at the unexposed regions is substantially unmodified to form a patterned surface on the substrate.

The method described herein is conducted with a patterned microplasma and does not require the use of a separate mask or template on or adjacent the substrate surface in order to form the pattern thereon.

In some embodiments, the method further comprises:
(c) treating the patterned surface with a binding agent that binds at the exposed localised regions.

The binding agent may be any atom, molecule, cell or other moiety that selectively binds (whether directly or indirectly) to at the exposed localised region. For example, the binding agent may be a biological agent such as a tissue, cell (eukaryotic or prokaryotic), virus, extra cellular component, protein, glycoprotein, carbohydrate, fat, polynucleotide (including DNA molecules, RNA molecules, micro RNA molecules), or biological fluid.

In a second aspect, the present invention also provides a substrate comprising a patterned surface produced by a method of the present invention.

In a third aspect, the present invention further provides a substrate comprising a patterned surface, wherein the patterned surface comprises a coating that has been modified at localised regions by selective exposure to patterned microplasma to form exposed localised regions and substantially unmodified unexposed regions, and wherein a binding agent is optionally bound at the exposed localised regions.

In a fourth aspect, the present invention also provides a use of a substrate of the second and third aspects of the present invention, in techniques selected from the group consisting of a protein binding assay, a biosensor, a microarray, a therapeutic vehicle, disease diagnosis, a sample collection device, a purification matrix, separation matrix, a biochip, a cell or tissue culture substrate, a biomaterials scaffold, a tissue engineering scaffold, a cell array, and a cell expansion surface.

### BRIEF DESCRIPTION OF THE FIGURES

Illustrative embodiments of the present invention will be discussed with reference to the accompanying Figures.

FIG. 1 provides (a) a schematic diagram of the microplasma array device, (b) a photograph of the microarray device, and (c) a photograph of and the microplasma array device during ignition (operated at 1 kVₚₑₐₖ₋ₚₑₐₖ and 10 kHz in helium at 760 Torr).

FIG. 2 provides ToF-SIMS images and line scans of microplasma array treated BSA-coated polystyrene surfaces: (a) ToF-SIMS image of BSA-related fragments (CH₂N⁺, CH₄N⁺, C₄H₈N⁺, and C₅H₁₀N⁺), (b) line scan graph of intensity shown along line indicated in (a); (c) ToF-SIMS image of polystyrene-related fragments (C₇H₇⁺ and C₉H₇⁺), (d) line scan graph of intensity shown along line indicated in (c); (e) overlaid image of ToF-SIMS images shown in (a) and (b) with BSA-related fragments shown in brighter contrast and polystyrene-related fragments shown in darker contrast, (f) overlaid images of line scan graphs of BSA-related (broken line) and polystyrene-related (solid line) fragments shown in (b) and (d), respectively; scale bar=1 mm.

FIG. 3 provides a graph of normalized intensities of positive fragments for BSA-coated polystyrene substrates after microplasma array treatment, wherein ToF-SIMS region of interest (ROI) spectra were acquired within microplasma-treated regions (open columns) and in the background area (closed columns) within the array.

FIG. 4 provides (a) a fluorescence micrograph of a polystyrene substrate coated with fluorescently-labelled BSA (Invitrogen) after microplasma array treatment, and (b) the corresponding fluorescence intensity profile across a section of the array, as indicated by the broken white line in (a).

FIG. 5 provides (a) a fluorescence micrograph of fluorescently-labelled streptavidin on BSA-coated polystyrene after microplasma array treatment, and (b) the corresponding fluorescence intensity profile across a section of the array, as indicated by the broken white line.

FIG. 6 provides (a) a fluorescence micrograph of fluorescently labelled streptavidin on microplasma array treated polystyrene in the absence of a BSA coating step, and (b) a micrograph showing the pattern of wettability on the array.

FIG. 7 provides ToF-SIMS images of PLL-g-PEG passivated polystyrene treated with the microplasma array for 60 s. Positive ToF-SIMS images of (A) PLL-g-PEG-derived fragment ions (C₂H₅O⁺, 45.04 amu and C₃H₆N⁺, 56.05 amu), (B) an image of polystyrene-derived fragment ion (C₇H₇⁺, 91.05 amu) and (C) an overlay image of PLL-g-PEG-derived (red) and polystyrene-derived (green) fragments in (A) and (B), respectively. Scale bar=1 mm. Microplasma operating parameters: 7×7 array of 250 μm cavities, applied voltage—950 Vₚₖ₋ₚₖ, frequency—10 kHz, sample-array separation distance—150 μm.

FIG. 8 provides a graph of normalised intensities of positive fragments of microplasma-treated PLL-g-PEG coating onto PS; (blue-coloured columns) microplasma treated regions and (red-coloured columns) background area. (Confidence intervals were calculated for P=95%).

FIG. 9 provides fluorescence micrographs of fluorescently-labelled streptavidin adsorbed onto (A) PS, (B) PLL-g-PEG/PS and (C) microplasma array treated PLL-g-PEG/PS for 60 s. Protein adsorption experiments were performed at room temperature (23±2° C.). Scale bar—500 μm. Microplasma operating parameters: 7×7 array of 250 μm cavities, applied voltage—950 Vₚₖ₋ₚₖ, frequency—10 kHz, sample-array separation distance—150 μm.

FIG. 10 provides ToF-SIMS images of DGpp/ODpp/SiO₂ coating after treatment with the microplasma array for 30 s. Positive ToF-SIMS images of (A) DGpp-derived fragment ion (C₃H₇O⁺, 59.06 amu) and (B) an overlay image of DGpp-derived (red) and a total image of a group of hydrocarbon fragments (C₂H₃⁺, 27.02 amu; C₂H₅⁺, 29.04 amu; C₃H₇⁺, 43.05 amu; and C₄H₇⁺, 55.05 amu) present in the ODpp survey spectra (green). Scale bar=1 mm. Microplasma operating parameters: 7×7 array of 250 μm cavities, applied voltage—950 Vₚₖ₋ₚₖ, frequency—10 kHz, sample-array separation distance—150 μm.

FIG. 11 provides a graph of normalised intensities of positive fragments of microplasma-treated DGpp/ODpp/SiO₂; (blue-coloured columns) microplasma treated regions and (red-coloured columns) background area. (Confidence intervals were calculated for P=95%).

FIG. 12 provides fluorescence micrographs of fluorescently-labelled streptavidin adsorbed onto (A) ODpp/SiO₂, (B) untreated DGpp/ODpp/SiO₂ and (C) microplasma array treated DGpp/ODpp/SiO₂ for 30 s. Protein adsorption experiments were performed at room temperature (23±2° C.). Scale bar—500 μm. Microplasma operating parameters: 7×7 array of 250 μm cavities, applied voltage—950 Vₚₖ₋ₚₖ, frequency—10 kHz, sample-array separation distance—150 μm.

FIG. 13 provides fluorescence micrographs showing microplasma array patterning of protein on microscope glass slides following BSA coating and microplasma array treatment, with (a) patterned fluorescently-labelled streptavidin and (b) the corresponding fluorescence intensity line scan across a section of the array, as indicated by the broken white line in (a).

FIG. 14 provides a brightfield micrograph of a portion of an HRP enzyme array on microplasma-treated BSA-coated polystyrene after incubation with TMB substrate.

FIG. 15 provides fluorescence micrographs showing specific immunorecognition of anti-GFP and/or anti-RFP for GFP and/or RFP, respectively, following binding of anti-GFP and anti-GFP to BSA-coated, microplasma array treated polystyrene substrate, with (a), (c), (e), (g) captured through a green fluorescence filter and (b), (d), (f), (h) through a red fluorescence filter, the “+” symbol indicates presence of the antibody and the “−” symbol represents absence of the antibody on the substrate, and all substrates were exposed to a mixture containing the target GFP and RFP analytes.

FIG. 16 provides fluorescence micrographs of spots of dried (a) GFP and (b) RFP on silicon wafer, showing fluorescence aggregates.

FIG. 17 provides fluorescence micrographs showing localised cell attachment and proliferation on microplasma-patterned BSA-coated polystyrene at (a) 4 h, (b) 24 h and (c) 48 h after cell seeding, cells are stained with hoescht 33342 (blue, nuclear) and Cell Tracker Orange (red, cytoplasmic) dyes.

FIG. 18 provides fluorescence micrographs of MSCs that had been cultured for 48 h on microplasma treated ODpp/THX coverslips for 10 s (A) and 30 s (B). Cellular actin was stained with Phalloidin-TRITC (red) and cellular nuclei were stained with DAPI (blue). Microplasma operating parameters: 7×7 array of 250 μm cavities, applied voltage—950 Vₚₖ₋ₚₖ, frequency—10 kHz, sample-array separation distance—150 μm.

FIG. 19 provides fluorescence micrographs of MSCs that had been cultured for 48 h on untreated BSAODpp/THX coating (A) and microplasma treated BSA/ODpp/THX coverslips for 30 s (A). Cellular actin was stained with Phalloidin-TRITC (red) and cellular nuclei were stained with DAPI (blue). Microplasma operating parameters: 7×7 array of 250 μm cavities, applied voltage—950 Vₚₖ₋ₚₖ, frequency—10 kHz, sample-array separation distance—150 μm.

FIG. 20 provides fluorescence micrographs showing an array of human lymphocyte cells on microplasma patterned BSA/PS/SiO₂ coating. The coating was treated with microplasma array for 30 s then incubated in an antibody solution (CD20, 1 mg/ml) for 8 h. The antibody patterned coating was then cultured lymphocyte B cells (P3HR1K Cells) overnight. Scale bar—500 μm. Microplasma operating parameters: 7×7 array of 250 μm cavities, applied voltage—900 Vₚₖ₋ₚₖ, frequency—10 kHz, sample-array separation distance—150 μm, treatment time—30 s.

FIG. 21 provides micrographs showing HeLa and SKNSH cancer cells attached to ETFE substrates after microplasma array treatment. Scale bar—100 μm. Microplasma operating parameters: 7×7 array of 250 μm cavities, applied voltage—900 Vₚₖ₋ₚₖ, frequency—10 kHz, sample-array separation distance—150 μm, treatment time—3 min.

FIG. 22 provides (a) (right) an optical micrograph section of the electrode/microchannel assembly of an integrated microplasma/microfluidic chip device for surface patterning of bonded microchannels and (inset on the left) the assembled chip ready for operation, (b) an optical micrograph of localised microplasma generation inside the microchannel during operation in helium, and (c) fluorescence micrograph of a microplasma-patterned microchannel after incubation with fluorescently-labelled streptavidin.

### DETAILED DESCRIPTION

The present applicant has developed a method of patterning a surface on a substrate using patterned microplasma exposure. This technique advantageously provides spatially controlled surface modification without using a physical mask that is in contact with the substrate, or additional photolithographic steps. Advantageously, this method reduces the use of environmentally harmful organic chemicals and expensive vacuum systems and reduces the number of processing steps. The present applicant has shown that the substrates patterned by the method of the invention can be used in assays that detect proteins, protein binding and enzymatic reactions. Moreover, the present applicant has shown that the substrates patterned by the method of the invention can be used in cell culture.

Accordingly, in a first aspect, the present invention provides a method of patterning a surface of a substrate comprising:

(a) applying a coating to the surface to form a coated surface, and

(b) treating the coated surface with a patterned microplasma comprising a plurality of localised microplasma discharges such that localised regions of the coated surface are selectively exposed to the localised microplasma discharges to form exposed localised regions and unexposed regions that have not been substantially exposed to a microplasma discharge;

wherein the coating at the exposed localised regions is modified by the patterned microplasma and the coating at the unexposed regions is substantially unmodified to form a patterned surface on the substrate.

Persons skilled in the art will understand that the term “patterning” in this context refers to a technique of controlling the positioning of agents on a surface. For example, patterning can relate to the positioning of agents such as molecules including biological molecules or even cells upon the surface. The patterning described herein is achieved by selectively exposing the coated substrate surface to the plurality of localised microplasma discharges simultaneously to form a pattern comprising exposed localised regions and unexposed regions.

The term “microplasma” as used herein refers to a plasma that is confined to small dimensions, for example, a volume of about 100,000 nm³ to about 10 cm³, for example, 10 μm³ to 10 mm³. In some embodiments, the volume of a microplasma may be 1 mm³ to 10 mm³. In some embodiments of the present invention, a microplasma source may be cylindrical with a depth of 55 nm and a diameter of 250 μm. Accordingly, a microplasma source may have a volume of approximately 11×10¹² nm³.

Persons skilled in the art will understand that a plasma is an electrically-excited ionized gas or gases, that, upon excitation (eg ignition), forms a highly reactive environment that can modify materials directly exposed to the plasma discharge. The microplasma of the present invention can be operated over a wide range of pressures (for example, from 10 mTorr to above atmospheric pressure (eg 10× atmosphere or higher)), however, it preferably is operated at atmospheric or near atmospheric pressure. The microplasma can be generated in a variety of inert gases, for example, neon, helium, xenon, argon and combinations thereof. In some embodiments, the microplasma may consist of a combination of an inert gas (eg helium, neon, argon, krypton, xenon, radon, sulphur hexafluoride, etc) and a reactive gas (eg air, oxygen, water, nitrogen, fluorine, chlorine, etc). In some specific embodiments, the microplasma is a helium microplasma. The microplasma can be operated at a range of frequencies (low-frequency direct current (DC) and alternating current (AC), pulsed DC, radio frequency (RF), and microwave) (Iza et al., 2008).

The term “patterned microplasma” as used herein, is intended to refer to a microplasma that effectively has a number of localised, discrete plasma discharges, or alternatively, a number of simultaneous microplasma discharges that each have a distinct source, such that the microplasma(s) effectively operate in a particular uniform or non-uniform manner providing there are areas between the microplasma discharges in which no (or markedly reduced) microplasma is present.

The source of the microplasma may be any suitable microplasma source known to persons skilled in the art, for example, microhollow cathode discharges, dielectric barrier discharges, RF inductively coupled microplasmas, RF capacitively coupled microplasmas, microwave microplasmas, microfluidic discharge devices, microplasma jets, microplasma arrays of electrodes and patterned microplasma array devices such as a microcavity array devices, providing that a patterned microplasma is produced (Iza et al., 2008).

The term “localised microplasma discharges” or “localised discharges” as used herein is intended to refer to a microplasma that effectively functions as a number of separate but simultaneous microplasma discharges or alternatively is a number of separate but simultaneous microplasma discharges.

In some embodiments, the microplasma may consist of a number of sources, each of which may be individually addressable.

The term “localised regions” as used herein is intended to refer to uniform or non-uniform areas on a surface that are separated by background, areas. Preferably, such localised regions are uniform. Preferably, such regions have well defined boundaries.

The term “selectively exposed” as used herein is intended to describe the action of the patterned microplasma, specifically, that the pattern of the microplasma dictates which areas of the surface are subjected to the modifying action of the patterned microplasma.

The substrate may consist of any suitable substrate known to persons skilled in the art, for example, glass and coated glass surfaces (eg glass slides), polymers such as polystyrene (eg polystyrene slides, polystyrene dishes, polystyrene coated materials eg polystyrene coated silicon wafers), polycarbonate, polyesters, silicon wafers, etc. In some embodiments, the substrate is a synthetic implantable material (also referred to as a “biocompatible material”), such as polyethylene (PE), polytetrafluoroethylene (PTFE), expanded polytetrafluoroethylene (ePTFE), polymethacrylate (PMMA), fluorinated ethylene-propylene (FEP), poly(ethylene-co-tetrafluoroethylene) (ETFE), perfluoroalkoxy (PFA), polyurethane (PU), cellulose, metals (eg stainless steel, titanium, etc), alloys, ceramics, etc. The substrate may have “open” (ie unenclosed) surfaces, for example, planar surfaces, curved surfaces etc. In some embodiments, the surface of the substrate is an open surface that is substantially planar. In another embodiment, the surface of the substrate is an enclosed surface, for example, the inner surface of a microchannel, etc. The microchannel may be part of a microfluidic chip, eg a “lab-on-a-chip” devices, point of care devices, etc.

In some embodiments, the surface of the substrate may be treated prior to application of the coating to assist with bonding of the coating to the surface. For example, in many applications it may be preferable for the coating to be covalently bonded to the substrate surface. In many cases the substrate surface will be devoid of functional groups suitable for covalent linking and, therefore, it may be necessary to modify the substrate to provide functional groups on the surface. Suitable substrate surface treatments include wet chemical treatments by oxidising solutions to produce polar surface groups (eg alcohol, carbonyl, acid, epoxy), gas surface oxidation, and plasma or corona oxidation (low pressure or high pressure). In some embodiments, the substrate surface is treated by plasma polymerisation, to provide a thin, highly adherent surface rich in polar functional groups. Suitable materials for this purpose include acids (from acid containing monomers e.g. acrylic acid plasma); amines (from allyl amine or an amine monomer); epoxy groups; and thiols. Amines can be created by plasma polymerisation of amine-based monomers. Examples of these monomers are allylamine, diaminocyclohexane, 1,3-diaminopropane, heptylamine, ethylenediamine, butylamine, propargylamine, and propylamine. The plasma polymerisation of acetonitrile or acrylonitrile may also be used to introduce nitrogen functionality onto the surface. Suitable reagents and methods for plasma polymerisation onto a surface to create functionality thereon are described in Siow, et al., 2006.

Alternatively, or in addition, the surface of the substrate may be treated with a silane prior to application of the coating. This may be particularly suitable for glass substrates. A range of silanes have been used to “silanize” glass and can be used for present purposes. An example of a suitable silane is 3-aminopropyl triethoxysilane (APTES).

The coating agent that is used in the application of the coating to the surface of the substrate may comprise any suitable coating known to persons skilled in the art. In some embodiments, the coating agent is a biological material. Suitable biological materials include proteins, carbohydrates, fats, polynucleotides, biological fluids, etc, or fragments or extracts thereof, or combinations thereof. In some embodiments, the coating agent is a biological molecule such as a protein, carbohydrate, fat, polynucleotide, etc, or fragment thereof, or combinations thereof. For example, the coating agent may be a biological molecule such as protein or fragments thereof (eg albumin such as human serum albumin, bovine serum albumin (BSA), casein, etc), glycoproteins (eg fibronectin), carbohydrates including sugars (eg sucrose, glucose, lactose, etc), allyl alcohol, dried milk powders, egg white extracts, etc. In some embodiments, the coating agent is BSA. In some embodiments, the coating agent may be recombinantly produced.

Alternatively, the coating agent may be a synthetic agent. Suitable synthetic coating agents include polymers such as glycol polymers (eg polyethylene glycol (PEG)), ene polymers (eg octadiene polymers), polysaccharides (eg dextrans, cellulose derivatives, agarose, alginic and hyaluronic acids), poly N-isopropylacrylamide (PNIPAM), dextrans, phosphocholines (neat and as copolymers), poly(hydroxethyl methacrylate), hyaluronic acid, pegylated SAMS, poly(hydroxyethyl) methacrylate (PHEMA), phosphorylcholine, poly(methyl-oxazoline) (PMOXA), graft copolymers of cationic polyelectrolyes (eg poly(L-lysine) or poly(ethylene imine)) and poly(ethylene glycol)), graft copolymers of cationic polyelectrolyes (eg poly(L-lysine) or poly(ethylene imine)) and dextran, poly N-vinyl pyrrolidone (pNVP), polyvinylpyrrolidone (PVP), polyvinyl alcohol, poly(hexamethylene disiloxane), tetraethylene glycol dimethyl ether (tetraglyme), triethylene glycol dimethyl ether (truglyme), diethylene glycol dimethyl ether (diglyme), poly(acrylic acid), polyacrylamide, fluoropolymers (eg PTFE, etc), poly vinyl pyrrolidone, etc. The coating may be a plasma polymer of any of the aforementioned coating agents. Plasma polymers of triglyme, tetraglyme, crown ethers, NVP, PVP and HEMA may be suitable.

The coating agent may be a monomer or pre-polymer precursor to any of the aforementioned coating agents and the coating may be formed on the surface of the substrate by polymerisation.

Other suitable synthetic coating agents include surfactants. Non-ionic surfactants may be particularly suitable. Suitable surfactants include Tween surfactants (eg Tween 20, titron X-100 etc), non-ionic block polymers, and pluronics.

Other suitable synthetic coating agents include silane coupling agents that may “block” surfaces, eg PEG silanes.

Other suitable synthetic coating agents include hydrogels, and hydrocarbons.

In embodiments, the coating is selected from the group consisting of bovine serum albumin (BSA), poly(L-lysine)-graft-poly(ethylene glycol) (PLL-g-PEG) copolymer, diethylene glycol dimethyl ether (diglyme) and octadiene plasma polymer(ODpp).

The coating may be applied to the surface of the substrate using any suitable coating method known to persons skilled in the art, for example, by immersion, flushing, plasma polymerisation, casting, spraying, spin coating or layer by layer deposition. Preferably, the coating is applied to the substrate as a substantially continuous coating. In some embodiments, the coating is applied to the surface of the substrate by immersion of or flushing of the surface with a solution comprising the coating agent to form the coated surface. The coating may be bonded to the surface by adherence, adsorption (including physisorption or chemisorption), covalent binding, non-specific binding, specific binding, etc. In some embodiments, the coating binds to the surface by non-specific binding.

In some embodiments, the coating may be bound to the surface via a first linker which binds to the substrate and to the coating. The linker may be grafted onto the substrate using photochemical grafting methods known to the person skilled in the art for the covalent linking of polymers and biomolecules to surfaces. Suitable linkers include photo-coupling reagents such as aryl azides, aryl diazarines, and benzophenone. Benzophenone has been reported to be one of the most efficient photophores. It can be activated via UV (for example, the UV originated from the microplasma source) at wavelengths (λ˜0.350 nm), that are expected to cause little damage to biomolecules. The linker can be pre-patterned on the surface, bind to a biopolymer or biomolecule, or it can be pre-mixed with a biomolecules/polymer before photoactivation processes. The photolinker can also be used for surface-initiated photopolymerisation (Marcon, et al., 2009; Lee, et al., 2008; Szunerits, et al., 2008; Pan, et al., 2004.)

The term “specific binding” as used herein is intended to refer to the bond between two molecules (eg proteins, peptides, polynucleotides, or fragments thereof) that each have a defined three dimensional structure, such that a particular region of the structure of the first molecule recognises and bonds with a particular region of the structure of the second molecule (eg the binding of a ligand to its receptor, or the binding or an antibody to the epitope to which it was raised). The term “non-specific binding” refers to the binding that occurs between two molecules in the absence of particular recognition between the structures of the molecules. Generally, specific binding occurs with higher affinity and/or avidity than non-specific binding.

As discussed previously, the substrate surface may be treated prior to application of the coating in order to “activate” the surface and make it suitable for covalent bonding with the coating agent. Plasma methods, including plasma treatments and plasma polymer depositions, which result in surfaces that contain amine, carboxy, hydroxy, aldehyde, sulfhydryl and epoxy groups may be particularly suitable. Suitable reagents and methods for plasma polymerisation onto a surface to create functionality thereon are described in Siow, et al., 2006.

Alternatively, or in addition, the substrate surface may be treated with an agent so as to functionalise the surface of the substrate with functional groups that are compatible with or react with the binding agent that is later bound to the exposed localised regions. En these embodiments, the coating is applied, as described in more detail below, and then it is selectively removed at the exposed localised regions to reveal the functional groups that are compatible with or react with the binding agent.

Spacer groups may also be used between the substrate surface (suitably functionalised) and the coating. For example, the substrate surface may be functionalised with aldehyde groups (as described previously) and a bifunctional polyamine may be used as a spacer group. One end of the difunctional polyamine can be reductively aminated with the aldehyde groups on the surface to tether it to the surface, whilst the second can be used to bind the coating agent.

Once the coating is formed, the coated surface is treated with the patterned microplasma. The action of each microplasma discharge modifies the exposed localised regions of the coated surface. The modification to the coating may be minor or major. For example, the coating may undergo a chemical change (eg oxidation), or the coating may be denatured or inactivated. Alternatively, the coating may be at least partially or completely removed or ablated. It is to be understood that the term “modified” as used herein to describe the effect of the microplasma discharge on the coating is intended to include a partial or complete removal of the coating in the exposed localised regions. For example, in some specific embodiments the coating at the exposed localised regions is substantially removed by the patterned microplasma.

In some embodiments, the coating is a passivating layer that prevents the surface of the substrate from binding the binding agent by “blocking” the surface. For example, the coating (eg at the unexposed regions) may inhibit the binding of the binding agent to the surface. However, in some embodiments, the modification of the coating at the exposed localised regions reduces the ability of the coating to inhibit binding of material at those regions. Thus, in some embodiments, the modification of the coating (ie at the regions that have been exposed to the microplasma) allows a binding agent to bind to the substrate at the exposed localised regions.

In some alternative embodiments, the coating is an “active layer” that enables or enhances the binding of the binding agent thereto. In these embodiments, the modification of the coating at the exposed localised regions reduces the ability of the coating to bind the binding agent at those regions.

The coating may be a single layer coating or a multi-layer coating. For example, a first coating may be applied to the substrate wherein the first coating binds in a specific manner with a second coating that provides passivation as described previously.

The present applicant has shown herein that binding agents such as proteins and cells will bind to the exposed localised regions of the patterned surface of the substrate. In some embodiments, the biological agent is selected from the group consisting of a protein, a sugar, a polynucleotide, and a cell. The protein may be, for example, an enzyme, a growth factor, an antibody, a peptide and/or a synthetic bioactive agent. The polynucleotide may be, for example, a DNA polynucleotide molecule, an RNA polynucleotide molecule, an mRNA polynucleotide molecule, an oligonucleotide molecule, or combinations thereof. The cell may be, for example, a mammalian cell, such as those derived from a cell line, or those derived directly from a mammal. Preferably, the cell is selected from the group consisting of: pluripotent cells such as stem cells, including embryonic, mesenchymal, and induced pluripotent stem cells; and mesenchymal progenitor cells (i.e. mesoblasts).

Thus, in some embodiments, the method further comprises:

(c) treating the patterned surface with a binding agent that binds at the exposed localised regions.

The binding agent may be any atom, molecule, cell or other moiety that selectively binds (whether directly or indirectly) to at the exposed localised region. By “selectively binds” we mean that the binding of the binding agent at the exposed localised region is greater than the binding at the unexposed regions of the substrate. The person skilled in the art will understand that there may still be some binding of the binding agent at the unexposed regions, but that the degree of binding at the unexposed regions is of such a low level that there is a practical difference in the degree of binding between the exposed localised regions and the unexposed region.

The binding agent may be a probe for use in a lab-on-chip or point of care diagnostic instrument. In this context, probes are typically molecules that selectively bind other, target molecules that are required to be detected.

In some embodiments, the binding agent is a biological agent. The term “biological agent” as used herein is intended to refer to any material of a biological origin whether synthesised, isolated from living organisms, in a native or modified form eg tissues; cells (eukaryotic or prokaryotic), viruses, extra cellular components, proteins, glycoproteins, carbohydrates, fats, polynucleotides (including DNA molecules, RNA molecules, micro RNA molecules), biological fluids, etc, or fragments or extracts thereof, or combinations thereof.

The biological agent may be any biological agent of interest, providing the target can bind directly or indirectly with the exposed localised regions. In some embodiments, the biological agent is any agent for which there is interest in assaying. For example, the biological agent may be selected from the group consisting of an analyte, a carbohydrate, a hormone, an enzyme, a reactant of an enzymatic reaction, a receptor, a ligand, a protein or peptide binding partner; an antibody, an antibody fragment comprising a specific binding portion, an epitope, an antigen, an aptamer, a polynucleotide, a microorganism (eg bacteria), a pathogen, etc. The person skilled in the art will understand that the biological agent can be utilised to directly or indirectly detect the presence of an assayable target molecule in a sample that specifically binds to the biological agent, for example, using specific antibodies or fragments thereof comprising specific binding regions, specific aptamers, specific binding partners, etc that are directly or indirectly detectable (eg using various labels known to persons skilled in the art such as fluorescent labels, radioactive labels, etc).

In some embodiments, the biological agent comprises at least one protein or fragment thereof that when bound at the exposed localised regions can specifically bind with a binding partner. The binding partner may be selected from the group consisting of a receptor, a ligand, a protein or peptide binding partner, an antibody, an antibody fragment comprising a specific binding portion, an epitope, an antigen, an aptamer, and a polynucleotide. In some embodiments, the biological agent is a mixture of proteins or fragments thereof, or combinations thereof.

In some embodiments, the biological agent comprises at least one required component for an enzymatic reaction. In this embodiment, when one required component for an enzymatic reaction is bound at the exposed localised regions, and any other required component(s) of the enzymatic reaction are provided under suitable conditions, the enzymatic reaction occurs. The occurrence of the enzymatic reaction can then be detected directly or indirectly using methods known to persons skilled in the art. For example, the enzymatic reaction may produce a measurable product, colour change, precipitate or other detectable agent.

Substrates patterned with proteins have previously been described to control the shape, position and behaviour of cells during cell culture. Patterning of proteins onto substrates for cell biological and related applications, e.g. for in vitro cell culture, may advantageously more closely replicate the spatial heterogeneity of molecules in the in vivo extracellular environment. Accordingly, the patterning of biological agents onto substrates may be useful for cell culture techniques. For example, a patterned substrate for cell growth may enhance the ability of stem cells to retain their multipotent or pluripotent form, which may permit stem cells to be expanded in vitro.

Accordingly, in some embodiments, the binding agent comprises a cell. The cell may be any cell known to persons skilled in the art providing it will adhere to the exposed localised regions of the patterned substrate. For example, the cell may be a mammalian cell, such as those derived from a cell line, or those derived directly from a mammal. In embodiments, the cell is selected from the group consisting of: stem cells, including embryonic, mesenchymal, and induced pluripotent stem cells; and mesenchymal progenitor cells (i.e. mesoblasts). In some embodiments, the biological agent is a multipotent or pluripotent cell.

Advantageously, the methods described herein may be particularly suitable for maintaining multipotency in mesenchymal stem cells (MSCs). To date, the clinical use of MSCs has been limited due to the propensity of the cells to spontaneously differentiate (with a concomitant reduction or loss in multipotency) in vitro. Recently, McMurray et al (2011) have developed a nanostructured substrate fabricated by electron beam lithography that retains stem cell phenotype and maintains stem cell growth over eight weeks. It has also been suggested that cell spreading influences the decision between self renewal and differentiation in ES cells and that cell shape and spreading respond to changes in adhesion to the substrate (Ingber 1997 and 1993). Accordingly, stem cells may be bound to the patterned surface formed using the methods described herein and, by reason of their inability to spread, the cells may retain phenotype for a period sufficient to allow them to be used in a clinical setting. Thus, the patterned substrate surface may be particularly useful as a cell expansion surface.

In other embodiment the binding agent comprises a cell of analytical interest, such as a cancer cell. In specific embodiments, the cell is selected from the group consisting of neuroblastoma cells, lymphocyte B cells, and human epithelial carcinoma cells.

The binding agent may be applied to the surface of the exposed localised regions using any suitable method known to persons skilled in the art, for example, by immersion, flushing, spin coating, lithographic printing, lithographic writing, contact pin printing, and ink jet printing. In some embodiments, the patterned surface is treated with the binding agent by immersion of or flushing of the patterned surface with a solution comprising the binding agent to achieve binding at the exposed localised regions of the surface of the substrate. The binding agent may be bound to the surface by adherence, adsorption (including physisorption or chemisorption), covalent binding, non-specific binding, specific binding, or may be bound to the surface via a linker, etc. In some embodiments, the binding agent is bound non-specifically at the exposed localised regions.

The patterned microplasma used herein is formed using a patterned microplasma source. Advantageously, the microplasma itself is patterned when it contacts, or is otherwise exposed onto, the coated surface of the substrate. Therefore, in contrast to some prior art methods, there is no need to use a mask in contact with the substrate to form the pattern thereon using the method of the present invention.

In some embodiments, the microplasma is formed between two electrodes wherein at least one of the electrodes is patterned. In this context, “patterned” means that there are at least two electrically conductive regions formed between the electrodes of the microplasma source such that a patterned microplasma comprising at least two discrete localised microplasma discharges is formed. As will be understood by the person skilled in the art, a microplasma discharge is only formed in a region between the two electrodes of the microplasma source. In the present case, one of the electrodes is patterned and, therefore, the microplasma discharge is predominantly formed between the electroactive regions of the patterned electrode (as opposed to the non-electroactive regions) and the counter electrode. The present inventors have found that such a patterned microplasma can generate tight, uniform and reproducible patterns of exposed localised regions on the substrate when it is exposed to the patterned microplasma without any substantial “halo” effects. In the case of a planar substrate, the coated surface of the substrate may be placed directly adjacent to the surface of the microplasma source, which is initiated so as to form the patterned microplasma and expose the coated surface to the patterned microplasma thus formed. The surfaces of the microplasma source and coated substrate are substantially parallel and the gap between these surfaces is typically 50-300 micrometers, more often 100-200 micrometers. Alternatively, a plurality of plasma jets (for example of a capillary design described in Iza, et al., 2008) may be used to create a patterned surface. Plasma jets may be “driven” under computer control to create features (eg geometric shapes) by CAD/CAM.

In the case of an enclosed surface (such as the interior surface of a microchannel), a plurality of electrodes may be positioned in a spaced arrangement along a length of the microchannel with a corresponding electrode similarly positioned in a spaced arrangement from the plurality of electrodes so that patterned microplasma can be formed within the channel and predominantly only between the electrodes. The microchannels may be part of a microfluidic chip, eg a “lab-on-a-chip”, point of care assay, etc. In some embodiments, the patterned microplasma source is integrated with a microfluidic chip.

An alternative method of forming a patterned microplasma for use in the method of the present invention is to mask at least one of the electrodes of the microplasma source with a patterned template. The patterned template may comprise a series of cavities through each of which localised microplasmas are generated. In some embodiments, the patterned template comprises a uniform array of cavities that mediate the formation of the patterned microplasma.

The patterned surface that is formed on the substrate may comprise a uniform array of exposed localised regions.

In some embodiments, a second linker may be used to assist binding of the binding agent at the exposed localised regions of the substrate. In some embodiments, the linker binds to the binding agent and may bond to the substrate at the exposed localised regions either covalently or conically.

Suitable linkers have been described previously herein in relation to the first linker and are also described in the literature (Marcon, et al., 2009; Lee, et al., 2008; Szunerits, et al., 2008; Pan, et al., 2004.).

In a second aspect, the present invention provides a substrate comprising the patterned surface produced by the method of the first aspect.

In a third aspect, the present invention provides a substrate comprising a patterned surface, wherein the patterned surface comprises a coating that has been modified at localised regions by selective exposure to patterned microplasma to form exposed localised regions and substantially unmodified unexposed regions, and wherein a binding agent is optionally bound at the exposed localised regions. Such a substrate may have some or all of the features described herein.

In a fourth aspect, the present invention provides a use of a substrate of the second and third aspects of the present invention, in techniques selected from the group consisting of a protein binding assay, a biosensor, a microarray, a therapeutic vehicle, disease diagnosis, a sample collection device, a purification matrix, separation matrix, a biochip, a cell or tissue culture substrate, cell expansion surface, a biomaterials scaffold, and a tissue engineering scaffold.

In some embodiments, a binding agent comprising a cell is bound to the exposed localised regions, wherein the cell is cultured on the substrate. In some embodiments, the cell is a stem cell characterised by multipotency or pluripotency, wherein the cell retains multipotency or pluripotency when cultured on the substrate.

### EXAMPLES

The invention is hereinafter described by reference to the following non-limiting examples and accompanying figures.

General

Surface Characterisation

Time-of-flight secondary ion mass spectrometry (ToF-SIMS) measurements were performed using a Physical Electronics Inc. PHI TRIFT V nanoToF instrument equipped with a pulsed liquid metal ⁷⁹Au⁺ primary ion gun (LMIG), operating at 30 kV. Surface analyses were performed using “bunched” Au₁ beam settings to optimize mass resolution. The instrument software's mosaic function was employed to collect image data over larger areas (mm scale). Spectra were collected in positive SIMS mode, typically using 100×100 micron raster areas. Experiments were performed under a vacuum of ≦3.8×10⁻⁸ Torr and in the static mode to minimize possible effects arising from sample damage.

Analysis of Means

A group of six positive ion ToF-SIMS spectra from regions of interest (ROI) were collected from microplasma-treated regions and from the background area, respectively. The spectra were processed by analysis of means with a group of positive ion fragments related to polystyrene (PS) and BSA, respectively (Table 1). The intensity of each fragment was normalised to the total counts of the selected fragments in each spectrum and the average was taken. The confidence intervals were calculated for p=95%. This methodology yielded statistical differences between the two groups of spectra based on a single variable (univariate) assessment.

TABLE 1
Positive fragments used in the evaluation of BSA treatment
with microplasma by analysis of means.

| PS-related | | BSA-related | |
|---|---|---|---|
| fragments | m/z | fragments | m/z |
| C₄H₃⁺ | 51 | CH₂N⁺ | 28 |
| C₆H₅⁺ | 77 | CH₄N⁺ | 30 |
| C₇H₇⁺ | 91 | C₄H₈N⁺ | 70 |
| C₉H₇⁺ | 115 | C₅H₁₀N⁺ | 84 |
| C₁₀H₈⁺ | 128 | C₇H₇O⁺ | 107 |
| C₁₃H₉⁺ | 165 | C₈H₁₀N⁺ | 120 |
| | | C₈H₁₀NO⁺ | 136 |
| | | C₁₀H₁₁N₂⁺ | 159 |
| | | C₁₁H₈NO⁺ | 170 |

Fluorescence Microscopy

Fluorescence imaging was carried out using a Nikon Inverted Microscope TE-2000 through a 4× objective. Images of the BSA-conjugated, microplasma array treated substrates were captured through a Nikon filter with 455-485 nm excitation and 500-545 nm emission. Images were recorded with a Nikon DXM1200C digital camera and processed using NIS-Elements Basic Research v2.2 software.

White-Light Optical Microscopy

Optical micrographs were acquired using a Nikon Eclipse LV150 optical microscope through a 5× objective and recorded with a digital Camera (DS-Fil, Nikon, Japan).

### Example 1

### Microplasma Patterning of Bovine Serum Albumin Coated Substrates

Polystyrene substrates were prepared as follows: A 5% (w/v) solution of polystyrene (Goodfellow Cambridge Ltd.) was prepared in toluene (Sigma). The solution was spin-coated onto polished silicon wafer pieces (Wafernet, Inc). The spin-coated samples were soft-baked at 50° C. for 5 min to facilitate the removal of residual toluene.

Glass substrates were prepared as follows: Commercial microscope glass slides (ProSciTech) were functionalised with 3-aminopropyl triethoxysilane (APTES, Sigma) to enhance protein adsorption onto the glass surface. The slides were incubated with an undiluted solution of APTES at 25° C. for 45 min, rinsed in isopropanol, dried under nitrogen and then soft-baked at 120° C. for 5 min.

Bovine serum albumin (BSA) coating was performed on the surface of the substrates (polystyrene or APTES functionalised glass slides) by incubating a 1% (w/v) solution of BSA (Sigma) in phosphate buffered saline (PBS, pH 7.4, Sigma) over the surfaces of the substrates at 25° C. for 4 h. The surfaces were then washed in Milli-Q water and dried under nitrogen.

For some experiments as described below, the same coating (passivation) protocol as above was used except that a 1% (w/v) solution of fluorescein-conjugated. BSA (Invitrogen) was incubated over the substrate at 25° C. for 4 h (instead of unlabelled BSA).

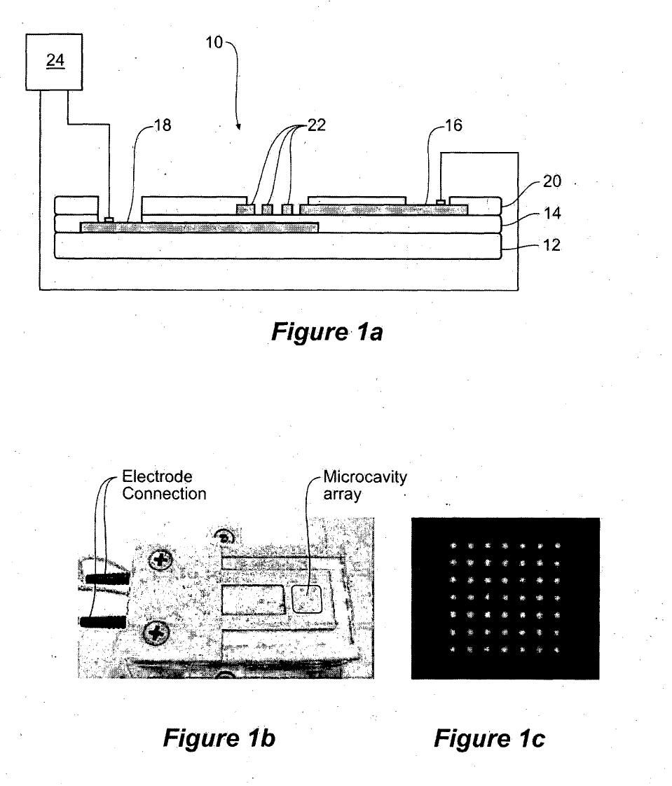

A 7×7 microcavity array patterned microplasma source was used as a representative example to demonstrate the capability of the source for localised surface treatment and thin film polymer deposition. A schematic representation of the device 10 is shown in FIG. 1(a). The device 10 is formed on a glass substrate 12 and an insulating dielectric layer 14 (SU8-50 photoresist, MicroChem Corp., USA) sandwiched between a top gold electrode 16 and a bottom gold electrode 18. A cover layer 20 was placed over the dielectric layer 14. A 7×7 array of 250 μm diameter cavities 22 with a depth of 55 nm and a separation distance (edge-to-edge) of 500 μm was patterned into the top gold layer using standard photolithography. Plasma generation was carried out using a custom-built electrical system 24. A power supply consisted of an oscillator (Agilent Technologies, DS06034A), an audio amplifier (AMPRO, XA1400) and a step-up transformer (Southern Electronic Services) powered the microplasma array using sinusoidal AC excitation.

The microcavity array patterned microplasma device 10 was fabricated using the following protocol: a Pyrex glass (H. A. Groiss & Co., Australia) substrate 12 was sonicated in isopropanol for one minute and subsequently cleaned by oxygen plasma for two minutes. On the glass substrate the first electrode (bottom) 18 was deposited by metal vapour deposition (MVD) (K975X High Vacuum Evaporation System, Emitech). It consisted of 5 nm chromium and 50 nm gold with the chromium functioning as an intermediate adhesion layer between the substrate and the gold. Then, a negative photoresist (SU8-50, MicroChem Corp., USA) was spin-coated (3000 rpm, 30 s) to form an insulation layer 14 (≈30 μm). It was sequentially baked (4 min at 65° C. and 15 min at 95° C.), exposed (EVG Mask Aligner, 270 mJ/cm2), post-exposure baked (1 min at 65° C. and 4 min at 95° C.), and developed. The photoresist was then hard-baked at 200° C. for 5 min. A second electrode (top) 16 (consisted of 5 nm chromium and 50 nm gold) was deposited on top of that by MVD. A positive photo resist (AZ 1518, MicroChemicals GmbH, Germany) was spin-coated and a 7×7 array of 250 μm diameter cavities 22 was patterned into the photoresist using a physical mask. The positive photoresist was hard-baked at 115° C. for 2 min. Etching of the electrode, using aqua regia (3:1 HCl:HNO₃) for the gold and ammonium cerium (IV) nitrate for the chromium formed the patterned array. After etching, the positive photoresist was completely removed with acetone. In the final step, the SU8-50 photoresist 20 was spin-coated over the entire surface to insulate the edges of the electrodes with the exception of the exposed gold electrode regions and the patterned area that were left blank.

The device 10 had an array of 49 cylindrical shaped cavities 22. Each cavity 22 was separated by 500 μm and had a diameter of 250 μm. The depth of each cavity was limited by the thickness of the top electrode 16, which was around 55 nm. Thus, the ignited device 10 gives an active area of 2.4 mm² spread over a treatable area of 22.56 mm². FIG. 1(b) shows a photograph of the microcavity array patterned microplasma source mounted on the top flange inside the chamber and FIG. 1(c) shows the device operating in atmospheric pressure helium gas. The microcavity array patterned microplasma source was fabricated using a photolithography fabrication method. Using this method, the number of cavities in the array can easily be upscaled or downscaled, in addition to the ability to fabricate different patterns and dimensions.

The microplasma experiment was carried out in a custom-built microplasma system designed and manufactured by Cantech Pty Ltd, Adelaide, Australia. A detailed description of the system is given elsewhere (Al-Bataineh et al., 2011; incorporated herein by reference). After the substrate was placed on the sample stage, the chamber was pumped down to a base pressure <5×10-2 Torr. It was then filled with high purity helium gas (99.99%, BOC) or a mixture of helium gas and 1,7-octadiene monomer (Alfa Aesar, Australia) to reach atmospheric pressure (760 Torr). For the latter experiment, the octadiene monomer was placed in a round-bottom flask and connected to the chamber by a stainless steel line and a manual flow control valve. The residual moisture in the monomer liquid was initially removed by several freeze-thaw pump cycles. Any volatile impurities present in the monomer line or chamber were removed by pumping on the monomer liquid for several minutes. The computerised stage was raised to bring the substrate close to the microcavity array device with a separation distance of approximately a few hundred microns, followed by the ignition of the microplasma source. The applied voltages, at frequency of 5 kHz, was 900 Vₚₑₐₖ₋ₚₑₐₖ for surface treatment. Each source in the array had the same plasma emission strength.

The microplasma source was operated at 1 kVₚₑₐₖ₋ₚₑₐₖ and 10 kHz in an atmospheric pressure (760 Torr) of helium. A photograph of the microcavity array microplasma source during ignition with helium is shown in FIG. 1 (c). The microcavity array microplasma source was mounted upside down on the top flange inside a custom-built microplasma system. Substrates (eg surface coated substrates) were placed face-up on an insulated sample stage for surface treatment with the microplasma array. The chamber was initially pumped down to a base pressure <5×10⁻² Torr to remove background air. For treatment, the chamber was filled with high purity helium (99.99%, BOC). A computerised stage was used to precisely control the distance between substrate and microplasma array, with the separation distance kept constant at 150 μm. The optimised treatment time for polystyrene and glass substrates was kept constant at 10 and 5 s, respectively. Each cavity of the array ignites discretely with very similar output, providing an array of spatially separated micro-compartments for the heterogeneous chemical modification of surfaces.

Polystyrene and glass substrates were coated with a BSA coating, and the BSA-coated surfaces were microplasma array treated in helium for 10 s. Each source or “cavity” in the array has the same microplasma emission strength. The microplasma treatment disrupted the BSA coating in an orderly fashion that produced uniform “wells” or “cavities” upon the substrate, in which the BSA coating was at least partially ablated or modified.

Inspection of the substrates following the coating and microplasma array treatment by optical microscopy and profilometry revealed no changes in surface topography were observable (data not shown).

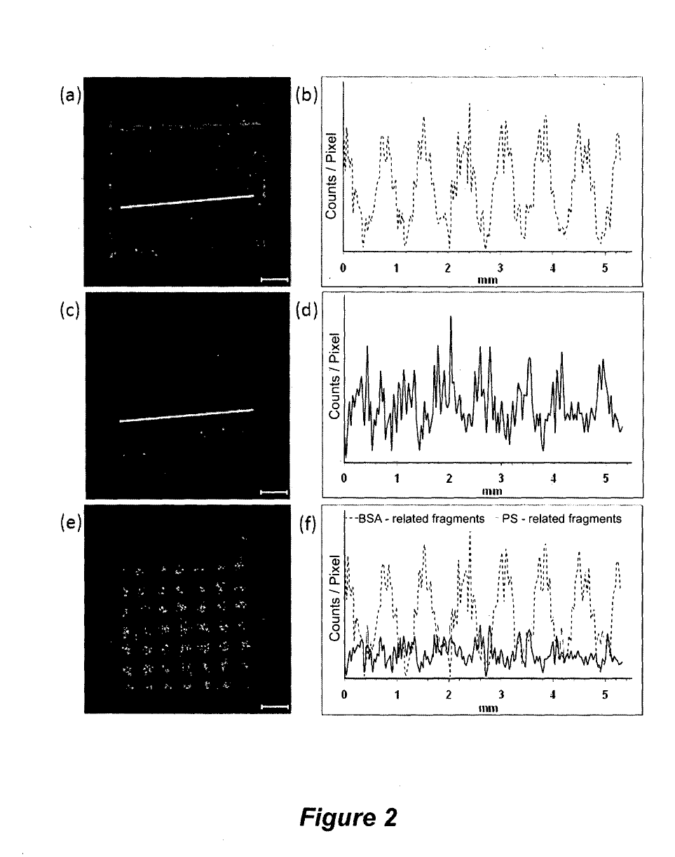

Static time-of-flight secondary ion mass spectrometry (ToF-SIMS) was used to image BSA distribution on the treated substrates following the microplasma exposure (FIG. 2). Imaging using a number of positive fragments (CH₂N⁺, CH₄N⁺, C₄H₈N⁺, and C₅H₁₀N⁺) that are characteristic of BSA revealed that the BSA protein was locally modified in the regions directly exposed to the microplasma emitted from each of the cavities of the array (FIG. 2(a)). A line scan, taken across a section of the treated substrate, indicated that each of the modified regions was uniformly treated (FIG. 2(b)). ToF-SIMS imaging and line scan measurements of selected positive fragments (C₇H₇⁺ and C₉H₇⁺) characteristic of the underlying polystyrene substrate (Davies et al., 2000) (C₇H₇⁺ is also expected from phenylalanine in BSA; Wald et al. 2010) are shown in FIGS. 2(c) and 2(d). From these images, it can be seen that there was a higher intensity of “polystyrene-type” fragments emanating from the microplasma-exposed regions, compared to the background (FIGS. 2(c) and 2(d)). This effect is clearly illustrated when the results derived from the hydrocarbon fragments from polystyrene were overlayed with the nitrogen-containing fragments from BSA (FIGS. 2(e) and 2(f)). Accordingly, FIG. 2 shows that the intensities of BSA-related fragments were significantly lower within the plasma-treated regions of the substrate compared to the background (ie non-microplasma treated regions). The opposite trend was observed for polystyrene characteristic fragments. Further, the results indicate that the microplasma treatment not only uniformly modified the BSA layer, but at least partially removed the BSA coating from the microplasma-exposed regions.

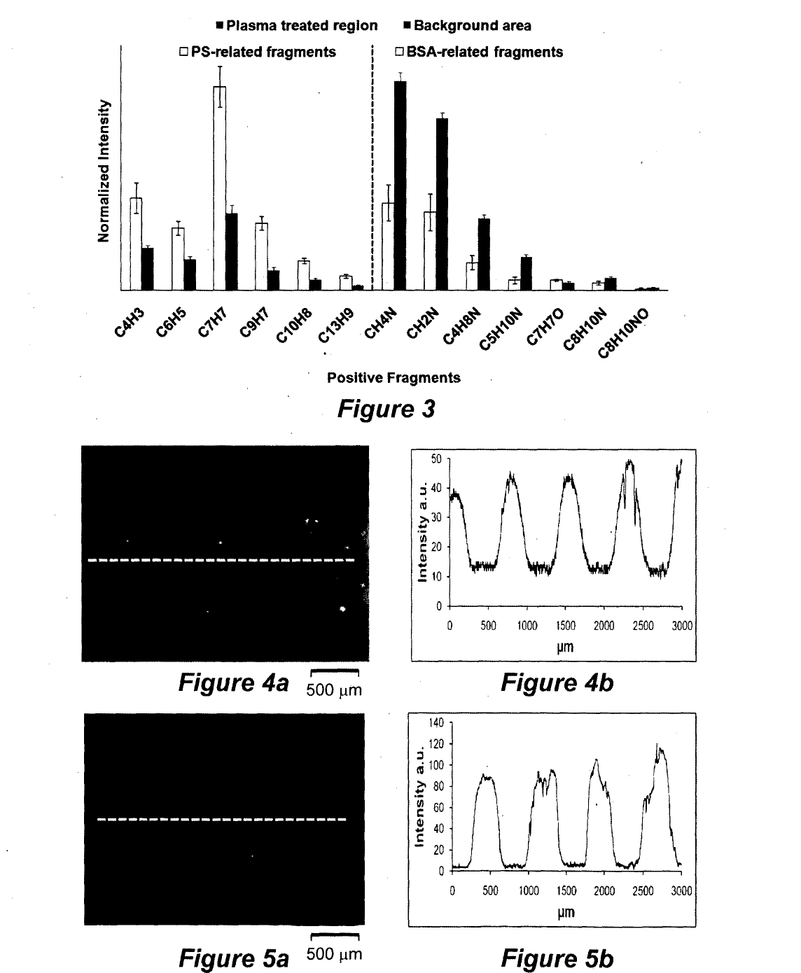

Further statistical analysis of the positive ToF-SIMS spectra supported the observation that the BSA coating was locally modified by microplasma array treatment. This methodology yielded statistical differences between the two groups of spectra (ie spectra from the microplasma-treated regions and the background area) based on a single variable (univariate) assessment (FIG. 3). FIG. 3 shows that the intensities of BSA-related fragments were significantly lower within the microplasma-treated regions compared to the background. The opposite trend was observed for the polystyrene (PS) characteristic fragments. The results from the analysis of means (FIG. 3) are consistent with the results presented by the ToF-SIMS images (FIG. 2).

Fluorescence microscopy was used to investigate the fluorescence of an adsorbed layer of fluorescently labelled BSA on polystyrene after microplasma array treatment. Consistent with the ToF-SIMS data, fluorescence was extinguished in the regions directly exposed to the microplasma treatment (ie in line with the cavities in the microplasma array; FIGS. 4a and 4b). The fluorescence signal had not recovered after 1 month, ruling out the possibility of photobleaching of the fluorophore. This result shows that the BSA was modified or at least partially ablated in the microplasma-exposed region.

Our data shows that during the microplasma treatment, the microcavity array device selectively exposed an array of localised regions of the BSA coated substrate to the microplasma, patterning the substrate surface by disrupting the BSA coating in an orderly fashion that produced uniform “wells” or “cavities” upon the substrate, in which the BSA coating was at least partially ablated or modified in a region-specific manner.

### Example 2

### Patterning of BSA Coated, Microarray Treated Polystyrene Substrates with Streptavidin Protein

BSA-coated substrates were microplasma array treated as described in Example 1. The substrate surfaces were incubated with 150 μl of 20 μg/ml Alexa Fluor® 568 conjugated streptavidin protein (Invitrogen, prepared in PBS) at 25° C. for 12 h. The surfaces were then washed with a solution of PBS containing 0.05% (v/v) Tween-20 (PBS-T, Sigma), rinsed in Milli-Q water and dried under nitrogen. The protein binding was visualized using fluorescence microscopy. A control was performed wherein the BSA coating step was omitted.

The microplasma array treated polystyrene substrate was also exposed to a stream of water vapour and the condensed water droplets were imaged with brightfield microscopy to determine the hydrated areas.

FIGS. 5a and 5b show the resulting contrast between regions of the substrate directly exposed to the cavities in the microplasma array and the surrounding background area, with the microplasma array exposed regions showing significantly higher fluorescence intensity than the non-exposed background. Accordingly, the protein streptavidin selectively bound to the regions of the substrate that had been exposed to microarray treatment (that is, the regions where the BSA protein had been modified or at least partially ablated by the microplasma treatment), but did not bind to the background regions which were not affected by the microplasma treatment (where BSA remained adsorbed). Further, the fluorescence intensity distribution inside each microplasma-modified region was similar, indicating a constant amount of protein binding across the array (FIG. 5b).

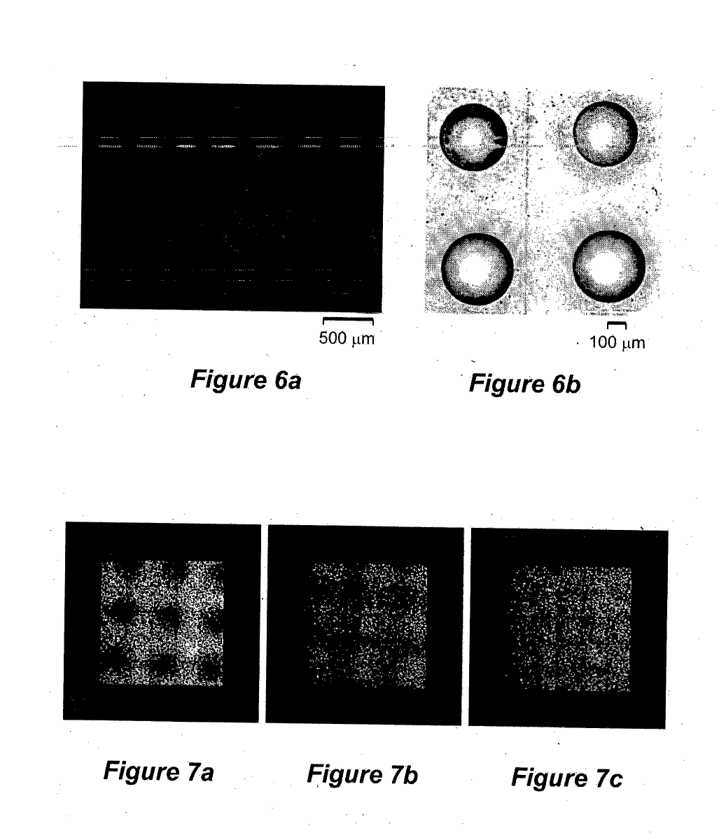

A control for the fluorescently-labelled streptavidin experiment was prepared by omitting the BSA coating step (FIG. 6(a)). The distribution of the streptavidin in the absence of the “blocking” BSA follows the pattern of wettability on the array (FIG. 6(b)), that is, proteins adsorbs preferentially to the hydrophobic regions (ie untreated polystryrene) rather than to the microplasma exposed, localized hydrophilic regions. Accordingly, the microplasma treatment created localised regions that adsorbed less protein.

### Example 3

### Patterning of PLL-g-PEG/PS Coated, Microarray Treated Polystyrene Substrates with Streptavidin Protein

Polystyrene (PS) substrates (GoodFellow, UK) were initially cleaned prior to poly(L-lysine)-graft-poly(ethylene glycol) (PLL(20)-g[3.5]-PEG(2)) copolymer (SuSoS AG, Switzerland) coating by rinsing in isopropanol, followed by drying under stream of nitrogen, and 5 min oxygen plasma. A 150 μl of 0.1 mg/ml polymer solution in HEPES II buffer (filtered through 0.2 μm membrane) was pipetted into each well and left to adsorb for at least 2 h at room temperature. The surfaces were then rinsed with HEPES II buffer followed by MilliQ water, blow-drying under a stream of nitrogen, and stored in clean containers. HEPES II buffer consisted of 150 mmol/l NaCl buffered with 10 mmol/l HEPES and adjusted to pH 7.4 by addition of 6 mmol/l NaOH.

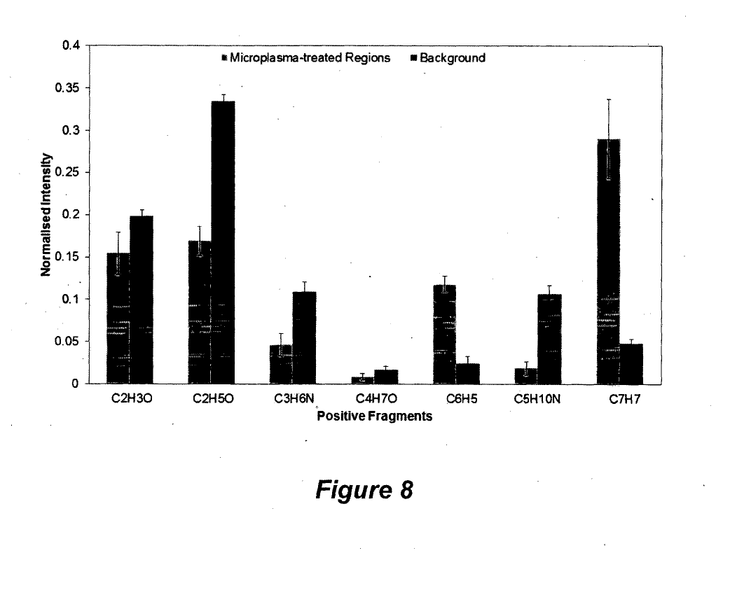

Static ToF-SIMS was used to image PLL-g-PEG coating post microplasma exposure. A positive fragment ions (C₂H₅O⁺, 45.04 amu and C₃H₆N⁺, 56.05 amu) characteristic of PLL-g-PEG (Pasche et al., 2003) revealed that the polymer was largely removed in the regions directly exposed to the microplasma cavities (FIG. 7A). A positive fragment ion (C₇H₇⁺, 91.05 amu) characteristic of the underlying PS substrate (Davies et al., 2000) was also imaged as shown in (FIG. 7B). From this it can be observed that there was higher intensity of PS characteristic fragments originating from the microplasma-exposed regions compared to the background. This effect is more evident when overlaying the PS and PLL-g-PEG images (FIG. 7C) and from statistical analysis (analysis of means) (FIG. 8).

Analysis of means (FIG. 8) revealed that positive fragments characteristic of PLL-g-PEG polymer such as C₂H₃O⁺ (43.02 amu), C₂H₅O⁺ (45.04 amu), C₃H₆N⁺ (56.05 amu), C₄H₇O⁺ (71.05 amu) and C₅H₁₀N⁺ (84.08 amu) were significantly reduced in intensity in the microplasma-exposed region compared to their normalised intensities in the background area. On the other hand, normalised intensities of positive fragments such as C₆H₅⁺ (77.04 amu) and C₇H₇⁺ (91.05 amu) characteristic to PS were substantially higher within the microplasma-treated regions compared to their normalised intensities in the background.

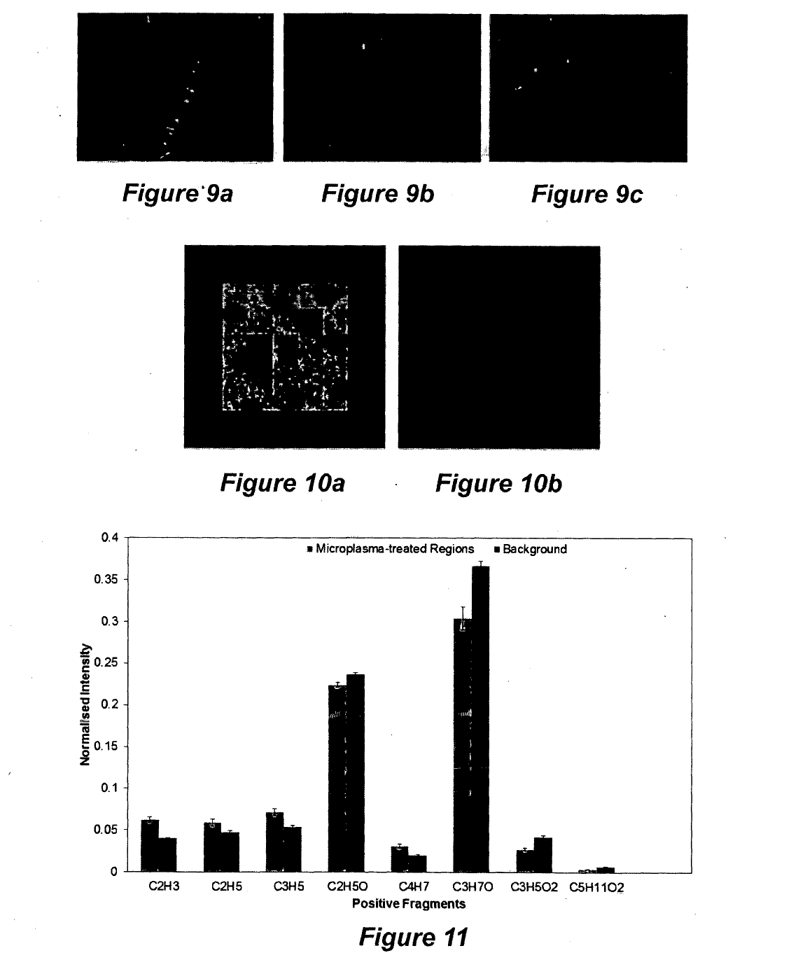

Upon exposure of microplasma treated PLL-g-PEG/PS coatings to fluorescently-labelled streptavidin at room temperature (23±2° C.), protein was locally adsorbed to microplasma-exposed regions (FIG. 9C). PS and PLL-g-PEG/PS surfaces (both untreated with microplasma) were also exposed to streptavidin solution at room temperature (as control samples). The fluorescence micrographs show that PS substrates support protein adsorption (FIG. 9A) however the surface exhibited non-fouling properties after coating a layer of PLL-g-PEG on top (FIG. 9B).

### Example 4

### Patterning of DGpp/ODpp/SiO₂ Coated Microarray Treated Silicon Substrates with Streptavidin Protein

Silicon wafer substrates (Siltron Inc., Korea) were ultrasonicated in isopropanol (AR grade, Merck) for 15 min and then dried using nitrogen gas. The monomers 1,7-octadiene and diethylene glycol dimethyl ether (diglyme) were both purchased from Sigma-Aldrich. Before plasma polymerisation, each monomer was processed with three freeze-thaw cycles to remove any dissolved gas. The plasma polymerisation process was carried out in a cylindrical chamber equipped with a radio frequency (13.65 MHz) generator. A full description of the system is described elsewhere (Zou et al., 2011). Ocatadiene (OD) monomer was introduced into the chamber at a flow rate of 1 standard cm³/min. A layer of OD plasma polymer (ODpp) was deposited on clean Si wafer substrates at a power of 10 W for 2 min and 5 W for 3 min, respectively. Diglyme (DG) monomer was introduced into the chamber at a flow rate of 0.8 standard cm³/min. A thin layer of DG plasma polymer (DGpp) was deposited onto the ODpp/SiO₂ samples at a power of 1 W for 10 min.

Static ToF-SIMS was used to image the DGpp/ODpp/SiO₂ coating (a PEG-like coating) after exposure to the microplasma array for 30 s. A positive fragment ion (C₃H₇O⁺, 59.06 amu) characteristic of the DGpp coating (Bretagnol et al., 2006) revealed that the polymer was largely removed in the microplasma exposed regions (FIG. 10A). An overlay image (FIG. 10B) of the ToF-SIMS image in FIG. 10A and a total ToF-SIMS image of a group of hydrocarbon positive fragments (C₂H₃⁺, 27.02 amu; C₂H₅⁺, 29.04 amu; C₃H₇⁺, 43.05 amu; and C₄H₇⁺, 55.05 amu) present in the survey spectra of the ODpp coating revealed that the intensity of the hydrocarbon fragments were higher in the exposed regions compared to the background. This effect is more evident from statistical analysis (analysis of means) (FIG. 11).

Analysis of means (FIG. 11) revealed that positive fragments characteristic of the deposited diglyme plasma polymer (DGpp) coating such as C₂H₅O⁺ (45.04 amu), C₃H₇O⁺ (59.06 amu), C₃H₅O₂⁺ (73.03 amu) and C₅H₁₁O₂⁺ (103.08 amu) were significantly reduced in intensity, in the microplasma-exposed regions compared to their normalised intensities in the background area. On the other hand, normalised intensities of the positive hydrocarbon fragments such as (C₂H₃⁺, 27.02 amu; C₂H₅⁺, 29.04 amu; C₃H₇⁺, 43.05 amu; and C₄H₇⁺, 55.05 amu) characteristic to the underlying ODpp layer were higher within the microplasma-treated regions compared to their normalised intensities in the background. This suggests that the ODpp coating was etched/damaged in the microplasma exposed regions, where remains non-fouling in the background region.

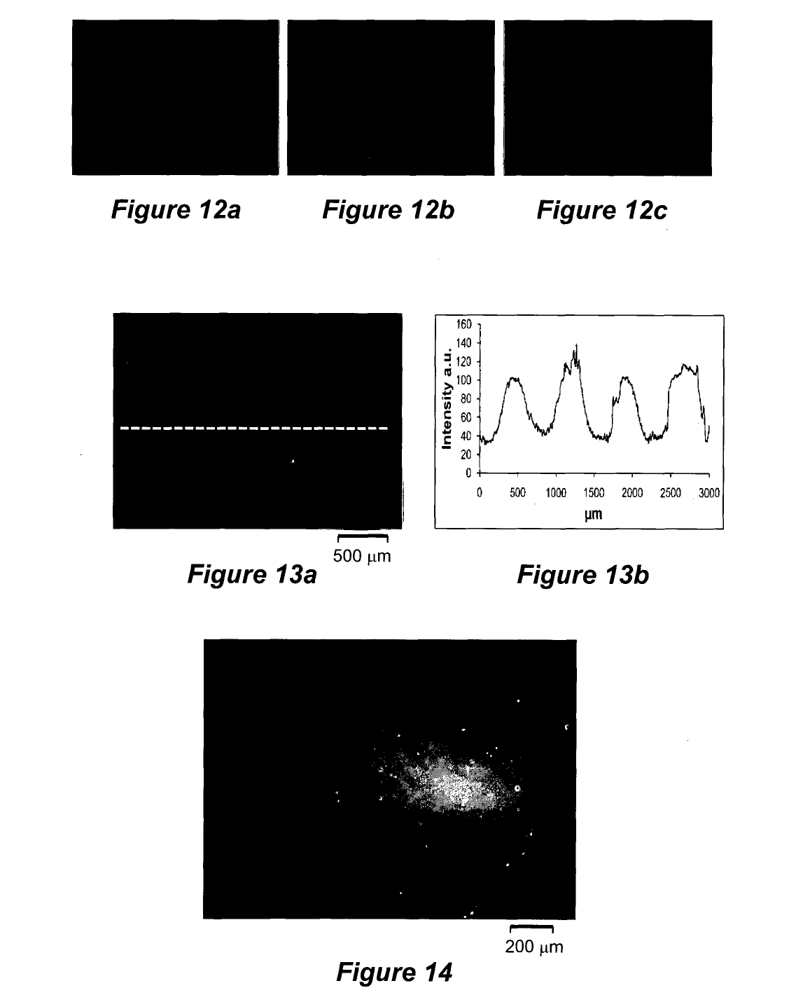

Upon exposure of microplasma treated DGpp/ODpp/SiO₂ coating to fluorescently-labelled streptavidin at room temperature (23±2° C.), protein was locally adsorbed to the microplasma-exposed regions (FIG. 12C). This indicates that microplasma array treatment of the DGpp coating for 30 s generated regions that support the adsorption of protein. Untreated ODpp/SiO₂ and DGpp/ODpp/SiO₂ coatings were also exposed to streptavidin solution at room temperature (as control samples). The fluorescence micrographs show that the ODpp coating support protein adsorption (FIG. 12A) however depositing a thin layer of PEG-like coating (i.e the DGpp layer) on top generated a surface that prevent protein adsorption (FIG. 12B).

### Example 5

### Patterning of BSA Coated, Microarray Treated Glass Substrates with Streptavidin Protein

The microplasma patterning method was also successfully applied to the patterning of proteins onto commercial silanized glass microscope slides. BSA coating and microplasma array treatment of glass slides enabled a constant amount of protein to be patterned across the array of a microscope glass slide. Specifically, a BSA coated, microplasma array treated, A PTES functionalised glass slide was incubated with fluorescently labelled streptavidin and then protein binding was visualised using fluorescence microscopy (see FIG. 13). FIG. 13 shows the resulting contrast between regions of the substrate directly exposed to the cavities in the microplasma array and the surrounding background area, with the microplasma array exposed regions showing significantly higher fluorescence intensity than the non-exposed background. Accordingly, the protein streptavidin selectively bound to the regions of the glass slide that had been exposed to microarray treatment (that is, the regions where the BSA protein had been modified or at least partially ablated by the microplasma treatment), in the same manner as for the polystyrene substrate, but the streptavidin did not bind to the background regions which were not affected by the microplasma treatment (where BSA remained adsorbed). Further, the fluorescence intensity distribution inside each microplasma-modified region was similar, indicating a constant amount of protein binding across the slide (FIG. 13(b)). The advantage of this approach is that the chemical and biological modification procedures do not macroscopically change the optical properties of the glass slide leaving it compatible for use with conventional microscope and high-throughput scanning instrumentation.

Passivation (or coating) of a polystyrene surface with an adsorbed layer of bovine serum albumin (BSA) provided a surface resistant to subsequent protein adsorption. The method of patterning surfaces using protein passivation followed by microplasma array treatment provides a straightforward and versatile means for mediating a regional-specific binding of biomolecules or bioentities such as proteins. The method permits protein binding from solution rather than from a printed drop, which advantageously increases the uniformity of protein deposition within each microplasma-treated region and less likelihood of denaturation and aggregation (Wu et al., 2008). This approach has applications in low-density microarrays for proteins and tissue growth experiments. Further, it improves the reproducibility of array dot fabrication compared with current approaches, potentially resulting in tighter data sets.

### Example 6

### Patterning of BSA Coated, Microarray Treated Substrates with Horseradish Peroxidase

BSA-coated substrates were microplasma array treated as described in Example 1. Horseradish peroxidase (HRP, Sigma) was used to determine the efficacy of binding of biologically active enzymes on the microplasma treated substrates. The microplasma cavity array patterned substrate surfaces were first incubated with 150 μl of 2.5 mg/ml HRP at 25° C. for 2 h, washed in PBS-T and then in PBS. A precipitating formulation of 3,3′,5,5′-tetramethylbenzidine (TMB, Sigma) was then incubated over the substrate surfaces at 25° C. for 10 min. The bioactive HRP oxidised the TMB, and the resulting precipitation of the oxidized TMB product was imaged by optical microscopy.

In this example, horseradish peroxidase (HRP) was adsorbed to the microplasma-patterned BSA surface in a region-specific manner, mimicking the format of an immunoblot assay. This surface was then incubated with a precipitating formulation of 3,3′,5,5′-tetramethylbenzidine (TMB). The localized HRP enzyme then catalyzed the oxidation of soluble and transparent TMB into a dark blue insoluble product that precipitated over the enzyme-containing regions (FIG. 14). The brightfield micrograph in FIG. 14 shows four distinct dark blue regions of precipitation caused by the HRP-catalysed oxidation of TMB at the microplasma-treated regions. This demonstrates that bioactive proteins such as enzymes can bind to microplasma treated regions with high intensity compared to the background regions and maintain their biological activity.

### Example 7

### Patterning of BSA Coated, Microarray Treated Substrates with Anti-GFP and Anti-RFP Antibodies

Microplasma array treated BSA-coated polystyrene substrates were prepared as described in Example 1. The substrate surfaces were either incubated with 150 μl of PBS as a negative control or with 10 μg/ml anti-GFP antibody (Rockland) in 150 μl of PBS at 25° C. for 12 h. The surfaces were washed in PBS-T and then in PBS. Next, the substrate surfaces were blocked with 150 μl of 1% (w/v) BSA-PBS solution at 25° C. for 2 h and washed as above. All samples were then incubated with 5 μg/ml GFP (Rockland) in 150 μl of PBS-T supplemented with 1% (w/v) BSA, at 25° C. for 2 h, and then washed in PBS-T and then in PBS. The binding of GFP to anti-GFP was visualised using fluorescence microscopy.

Microplasma array treated BSA-coated polystyrene substrates were prepared as described In Example 1. The substrate surfaces were either incubated with 150 μl of PBS as a negative control or with 10 μg/ml anti-RFP antibody (Rockland) in 150 μl of PBS at 25° C. for 12 h. The surfaces were washed in PBS-T and then in PBS. Next, the substrate surfaces were blocked with 150 μl of 1% (w/v) BSA-PBS solution at 25° C. for 2 h and washed as above. All samples were then incubated with 5 μg/ml RFP (Rockland) in 150 μl of PBS-T supplemented with 1% (w/v) BSA, at 25° C. for 2 h, and then washed in PBS-T and then in PBS. The binding of RFP to anti-RFP was visualised using fluorescence microscopy.

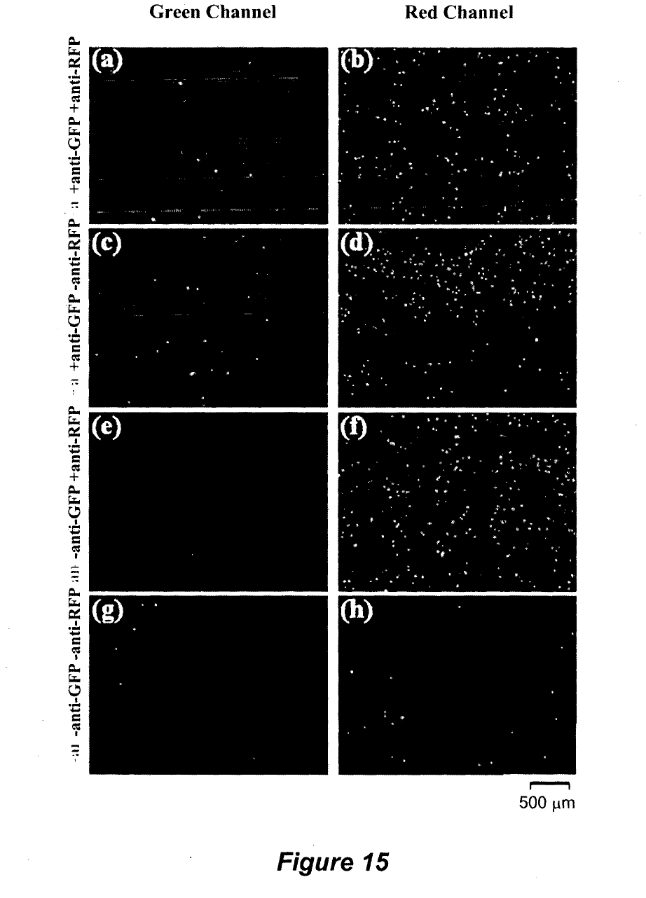

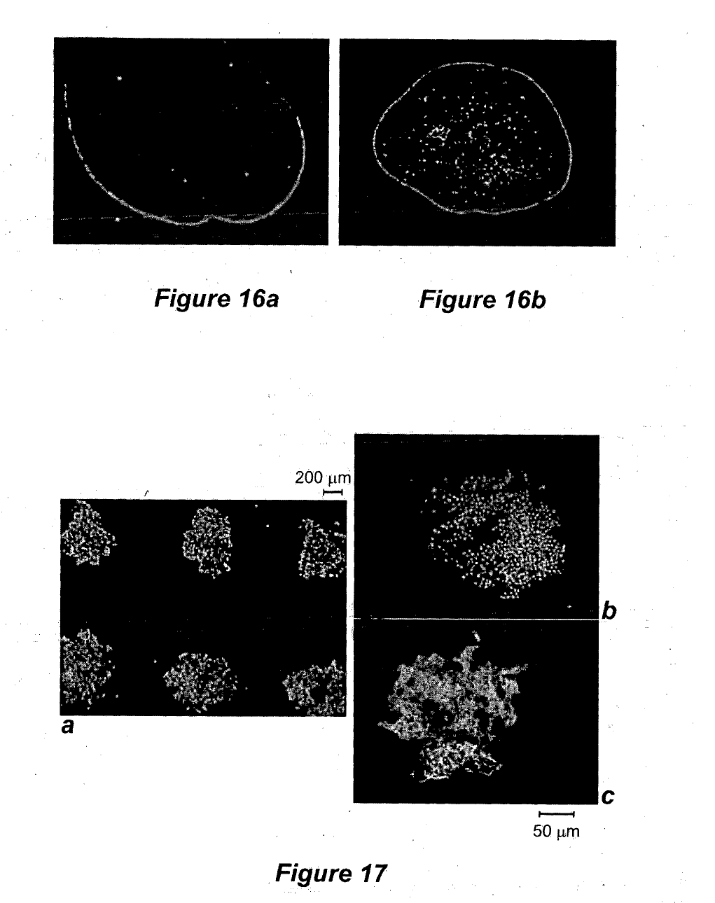

This example demonstrates that coated microarray treated substrates are suitable substrates for detecting multiple proteins simultaneously, indicating they are useful for the development of higher throughput immunoassays. The BSA-coated, microplasma-treated polystyrene substrate was functionalized as described above but with a mixture of antibodies specific for green fluorescent protein (GFP) and red fluorescent protein (RFP), that is, anti-GFP and anti-RFP antibodies were incubated with the substrate which facilitated binding of the antibodies to the microplasma-treated regions of the substrate. As shown in FIGS. 15(a) and 15(b), both GFP and RFP could be simultaneously captured and detected on the chip functionalised with both antibodies. Alternatively, the chip could be used for detection of only one target analyte. As shown in FIG. 15(c)-15(0, GFP or RFP could be specifically detected on the chips functionalised with either anti-GFP or anti-RFP antibody, respectively, with no non-specific protein binding evident from the non-target protein. GFP and RFP did not bind to the negative control, in which the antibody binding step was omitted. (FIGS. 15(g) and 15(h)). Fluorescent “speckles” in the background were present particularly on the images taken through the red channel. This was due to RFP aggregates present in the protein solution supplied by the manufacturer (see FIG. 16(b)), which did not appear to hinder the performance of the device for protein detection. These aggregates were not as notable in the GFP solution (see FIG. 16(a)).

### Example 8

### Culture of Human SK-N-SH Neuroblastoma Cells on Microplasma Patterned Substrates

Human SK-N-SH neuroblastoma (ATCC CRL-1573) cells were cultured in Dulbecco's Modified Eagle Medium (DMEM) (Sigma) supplemented with 2 mM L-glutamine, 100 IU/mL penicillin, 100 μg/ml streptomycin sulphate (Invitrogen) and 10% v/v fetal bovine serum (Sigma) and maintained at 37° C. in 5% CO₂. Prior to use with microplasma-patterned surfaces, cells were stained with CellTracker Orange CMRA (Invitrogen) as per the manufacturer's protocol.

Microplasma array treated BSA-coated glass slide substrates were prepared as described in Example 1. To assess cell attachment and growth, substrates were first incubated in culture media in 12 well culture dishes (Iwaki) for 10 minutes. Next, cells were seeded into the wells at a density 1×10⁵ cells/cm² and were cultured in contact, with microplasma-patterned substrates under standard culture conditions. Substrates were monitored at 4, 24 and 48 h. After incubation, substrates were washed gently with Dulbecco's phosphate buffered saline with calcium and magnesium (D-PBS+ Ca²⁺/Mg²⁺, 0.9 mM CaCl₂, 2.67 mM KCl, 1.47 mM KH₂PO₄, 0.50 mM MgCl₂-6H₂O, 138 mM NaCl, 8.10 mM Na₂HPO₄). Cells were fixed with 3.7% formaldehyde solution for 10 minutes, then stained with 2 μg/mL Hoechst 33342 (Sigma) in culture media for 10 minutes. Substrates were finally washed with D-PBS+ Ca²⁺/Mg²⁺ and mounted for analysis. Mounted substrates were imaged using an Eclipse50i fluorescence microscope (Nikon) with a DS-U2 digital camera (Nikon). CellTracker Orange CMRA was observed through excitation filter 540-557 nm and emission filter 605-625 nm and Hoechst 33342 through excitation filter 340-380 nm and emission filter 435-485 nm. All images were processed and analysed by NIS-Elements BR 3.0 software.

This example demonstrates that BSA coated, microplasma-patterned substrates can be used to create cell microarrays of attachment-dependent cell lines, such as those commonly used for drug screening (Keller et al., 2005; Wickstrom et al. 2007; Ekwall and Sandström, 1978; Hook et al. 2006). Both SK-N-SH (human neuroblastoma line) cells (FIG. 17(a)) and HeLa (human epithelial carcinoma line derived from cervical cancer) cells specifically attached to microplasma-treated regions, with minimal cell attachment to the BSA-coated background by 4 h post cell seeding. By 48 h following cell seeding, three-dimensional cell morphology typical of the SK-NH line developed within each microplasma-modified region (FIGS. 17(b) and 17(c)).

These cellular arrays could advantageously be utilised in screening protocols, such as identification of drug targets, where a moderate number of experimental replicates are required.

### Example 9

### Culture of Mesenchymal Stem Cells on Microplasma Patterned Substrates

Thermanox (THX) coverslips (Thermo Fisher Scientific, Australia) were rinsed in isopropanol (AR grade, Merck) and dried using nitrogen. The 1,7-octadiene monomer was purchased from Sigma-Aldrich. Before plasma polymerisation, the monomer was processed with three freeze-thaw cycles to remove any dissolved gas. The plasma polymerisation process was carried out in a cylindrical chamber equipped with a radio frequency (13.65 MHz) generator. A full description of the system is described elsewhere (Zou et al., 2011). Ocatadiene (OD) monomer was introduced into the chamber at a flow rate of 1 standard cm³/min. A layer of OD plasma polymer (ODpp) was deposited on the coverslips at a power of 5 W for 15 min.

BSA/ODpp/THX coatings were prepared by treating the ODpp/THX substrates with BSA as described in Example 1.

Murine mesenchymal stem cells (MSCs) cell line C3H/10T1/2 were grown to confluence, trypsinised and plated at 4×10³ cells per sample per well in basic basal media. Samples were then fixed and stained after incubating for 48 hours in 37° C., 5% CO₂. Briefly, cells were fixed in neutral buffered formalin, rinsed in sterile PBS, permeabilised with 0.2% TWEEN20/PBS, blocked with 1% BSA/PBS and incubated with 50 ug/ml Phalloidin-TRITC for one hour. Samples were then rinsed with sterile PBS and incubated for a further 3 minutes with 1 μg/ml DAPI in PBS, rinsed and cover-slipped using fluorescent mounting media. All cover-slipped slides were stored in the dark at 4° C. until required. Multiple images of each sample were taken at 200× magnification and captured using image analysis software, cellSens (Olympus).

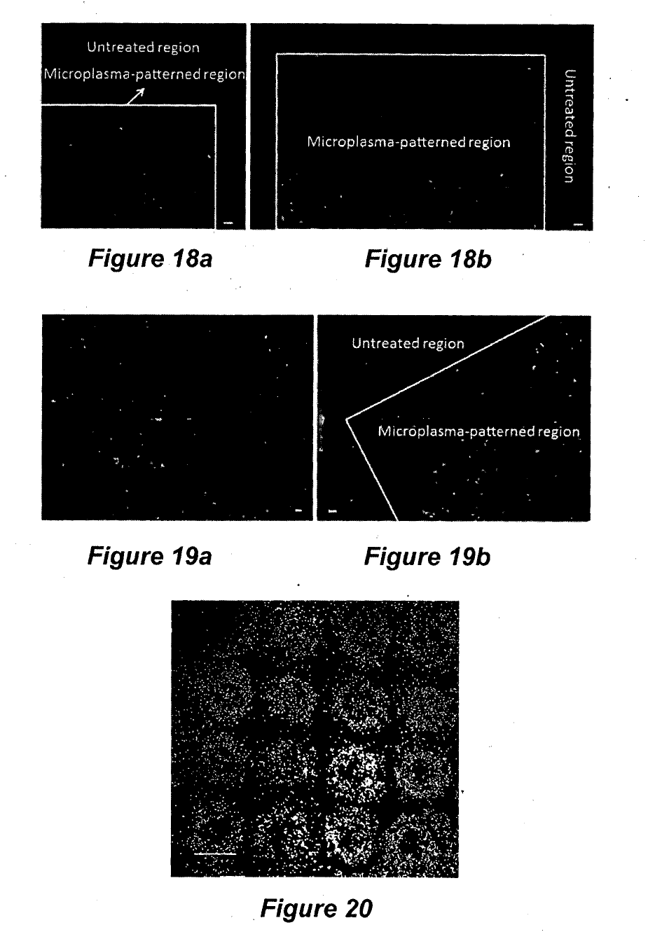

After microplasma array treatment of ODpp/THX for 10 s, MSCs were confined to the microplasma array exposed regions forming an array of cells (FIG. 18A). After 30 s of microplasma array treatment, the cells were attached homogeneously across an area that is confined to the size of the array and not to the individual cavities (FIG. 18B). For the BSA/ODpp/THX coatings, cells were attached homogeneously across the BSA passivated surfaces before and after microplasma array treatment for 30 s (FIGS. 19A and 19B). However, the cells were concentrated more over the microplasma treated region compared to the surrounding area. This data shows that generating an array of MSCs cell was possible on ODpp coatings after microplasma treatment for 10 s (FIG. 18A). Increasing the treatment time to 30 s led to the formation of a homogenous cell layer and the patterning was lost (FIG. 18B). This means that controlling the length of microplasma treatment for the ODpp coating is important. On the other hand, microplasma treatment of the BSA/ODpp/THX coating for 30 s encouraged more MSCs to adhere over the array-size treated region but not forming an array (FIG. 19B).

### Example 10

### Attachment of Lymphocyte B Cells to Microplasma Patterned Substrates

Anti-CD20 (Rituximab) was diluted in Dulbeccos phosphate buffered saline (PBS; pH=7.4) to 1 mg/ml. The microplasma patterned BSA coatings were submerged in the antibody solution for 8 h. The samples were extensively rinsed in PBS followed by a brief rinse in MilliQ water. The samples were dried in ambient conditions before incubation with lymphocyte B cells (P3HR1K Cells). The cells were grown in RPMI media (Invitro Technologies) with 10% FBS (Sigma) and 1% L-glutamine/penicillin/streptavidin (Sigma). A 2 ml aliquot of cell suspension was pipetted onto each array (cell seeding density of 2×10⁶ cells/ml) and incubated for 2 h at 37° C. After which, the arrays were rinsed in PBS to remove any unbound cells and fixed with 3.7% formaldehyde (Sigma-Aldrich) in PBS. The cells were stained with Hoechst (Invitrogen). The patterned samples were imaged on an Olympus IX81 inverted fluorescence microscope.

After microplasma treatment of BSA/PS/SiO₂ coatings for 30 s, the patterned coatings were submerged in the antibody solution for 8 h. After rinsing and drying, lymphocyte B cells (P3HR1K Cells) (normally not surface adherent) were cultured overnight in contact with the antibody patterned BSA/PS/SiO₂ coatings. The results show localised cell attachment (FIG. 20), meaning a higher number of functional antibody molecules adsorbed on the microplasma modified regions in comparison to the untreated BSA background, which remained undamaged and blocked the adsorption of the antibody molecules.

### Example 11

### Attachment of SKNSH and HeLa Cells to Microplasma Patterned Ethylene Tetrafluoroethylene (ETFE) Substrates

Human SK-N-SH neuroblastoma (ATCC CRL-1573) and HeLa (human epithelial carcinoma line derived from cervical cancer) cells were cultured in Dulbecco's Modified Eagle Medium (DMEM) (Sigma) supplemented with 2 mM L-glutamine, 100 IU/ml penicillin, 100 μg/ml streptomycin sulfate (Invitrogen) and 10% v/v fetal bovine serum (Sigma) and maintained at 37° C. in 5% CO₂. Cells were cultured in contact with microplasma-patterned ETFE samples at a density of 1×10⁵ cells/cm² and at 37° C. in 5% CO₂ in 12-well tissue culture dishes (Iwaki). After 24 h incubation, samples were washed gently with Dulbecco's phosphate buffered saline with calcium and magnesium (D-PBS+ Ca²⁺/Mg²⁺, 0.9 mM CaCl₂, 2.67 mM KCl, 1.47 mM KH₂PO₄, 0.50 mM MgCl₂-6H₂O, 138 mM NaCl, 8.10 mM Na₂HPO₄). Cells were fixed with 3.7% formaldehyde solution for 10 minutes, then stained with 2 μg/ml Hoechst 33342 (Sigma) in culture media for 10 minutes. Samples were finally washed with D-PBS+ Ca²⁺/Mg²⁺ and mounted in Fluoro-Gel/Tris buffer (ProSciTech) for analysis. Mounted samples were observed on an Eclipse50i fluorescence microscope (Nikon) with a DS-U2 digital camera (Nikon). Hoechst 33342 through excitation filter 340-380 nm and emission filter 435-485 nm. All images were processed and analyzed by NIS-Elements BR 3.0 software.

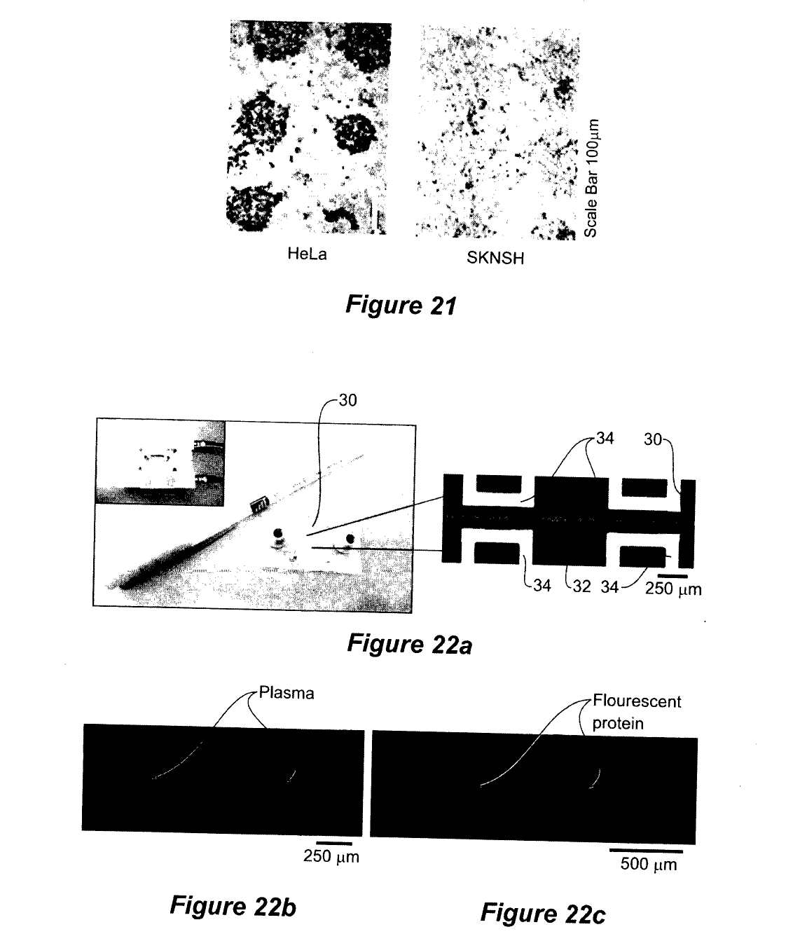

The microplasma patterning method was also used to pattern fluorinated polymeric substrates such as ethylene tetrafluoroethylene (ETFE) to create cell microarrays of attachment dependent cell lines. Specific cell attachment to the treated regions was observed for both SKNSH (human neuroblastoma line) and HeLa (human epithelial carcinoma line derived from cervical cancer) cell lines (FIG. 21). Antibody mediation was not required for cell adhesion to the array pattern. The results show that the cells were preferentially adhering to the hydrophilic pattern generated by microplasma treatment.

### Example 12

### Patterning of BSA Coated Enclosed Microchannels within a Glass Microfluidic Chip

Glass microfluidic chips 30 were prepared using a combination of UV-photolithography and deep-reactive ion etching (DRIE). Pyrex™ plates were spin-coated (2000 rpm) with SU8-10 photoresist and baked on hotplates for 2 min and 5 min at 65° C. and 95° C., respectively. The sample was then exposed (180 mJ/cm2, 360 nm) through a chrome-glass photomask patterned with the microchannel 32, and post-exposure baked for 1 min and 3 min at 65° C. and 95° C., respectively. The pattern was developed in the photoresist in SU8 developer solution for 3 min, was rinsed in isopropanol, and hard-baked for 1 min and 5 min at 95° C. and 150° C., respectively. DRIE (ULVAC NLD570) was carried out using fluorocarbon plasma (C4F8) at an etch rate (in Pyrex™ glass) of ˜0.3 mm/min. The final etch depth was 18 mm. Integration of the electrodes 34 into the glass microchip was carried out using molten gallium metal according to the methodology described in Priest et al., 2010.

The microchannel 32 wall was first functionalised with 3-aminopropyl triethoxysilane (APTES) by incubation with 100 mM 3-aminopropyl triethoxysilane (APTES), prepared in toluene at 25 C for 1 h. The microchannel was rinsed in toluene and then dried under nitrogen. The microchannel 32 was then coated by incubating with 1% (w/v) BSA prepared in PBS at 25□C for 4 h. The microchannel 32 was then flushed with Milli-Q water and dried under nitrogen. Microplasma array treatment was performed at 5 kVₚₑₐₖ₋ₚₑₐₖ and 10 kHz in a helium flow of 5 ml/min, wherein an embedded array of electrodes 34 was used to locally ignite several microplasma discharges along the length of the microchannel (FIGS. 22(a) and 22(b)). The design and operation of microplasma sources inside microfluidic chips has been described elsewhere (Priest., et al., 2010). A solution of 20 μg/ml Alexa Fluor 568 conjugated streptavidin protein (in PBS) was incubated in the microchannel at 25□C for 12 h. The microchannel was flushed with PBS-T, then Milli-Q water and finally dried under nitrogen. The protein was visualized using fluorescence microscopy as described above.

The fluorescently labelled strepatavidin protein was able to selectively adsorb to the microplasma exposed regions inside the chip (FIGS. 22(b) and 22(c)). This result shows the contrast between regions of the substrate directly exposed to the microplasma array treatment and the surrounding background area, with the microplasma array exposed regions showing significantly higher fluorescence intensity than the non-exposed background areas. Accordingly, the protein streptavidin selectively bound to the regions of the glass slide that had been exposed to microarray treatment (that is, the regions where the BSA protein had been modified or at least partially ablated by the microplasma treatment), in the same manner as for the polystyrene substrate, but the streptavidin did not bind to the background regions which were not affected by the microplasma treatment (ie where BSA remained adsorbed).

The present applicant believes this is the first disclosure of patterning of an enclosed surface.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before, the priority date of each claim of this application.

### REFERENCES

Al-Bataineh, S. A.; Britcher, L. G.; Griesser, H. J., Surf Sci., 2006, 600(4), 952-962.
Al-Bataineh, S. A., E. J. Szili, A. Mishra, S.-J. Park, J. G. Eden, H. J. Griesser, N. H. Voelcker, R. D. Short, D. A. Steele, Plasma Processes Polym. 2011, 8, 695-700.
Bretagnol et al., Plasma Processes and Polymers, 2006, 3, 443-455
Davies, J.; Nunnerley, C. S.; Brisley, A. C.; Sunderland, R. F.; Edwards, J. C.; Kruger, P.; Knes, R.; Paul, A. J.; Hibbert, H. Colloids Surf. Physicochem. Eng. Aspects 2000, 174, 287-295.
Ekwall, B. and B. Sandström, Toxicology Letters, 1978. 2(5): p. 285-292.
France, R. M.; Short, R. D. J. Chem. Soc., Faraday Trans. 1997, 93, 3173-3178.
Hook, A. L., et al., Trends Biotechnol, 2006. 24(10): p. 471-7.
Ingber D E. Annual review of physiology. 1997; 59:575-99.
Ingber D E. Gravit Space Biol Bull. 1997 June; 10(2):49-55.
Ingber D E. Journal of cell science. 1993 March; 104 (Pt 3):613-27.
Iza, F., et al., Plasma Processes Polym., 2008, 5, 322-344.
Keller, J., et al., Neurochemistry International, 2005. 46(4): p. 293-303.
Kreitz, S.; Penache, C.; Thomas, M.; Klages, C. P. Surf Coat. Technol. 2005, 200, 676-679.
Klages, C.-P.; Hinze, A.; Lachmann, K.; Berger, C.; Borris, J.; Eichler, M.; von Hausen, M.; Zänker, A.; Thomas, M. Plasma Processes Polym. 2007, 4, 208-218.
Lee, W., et al., Sensors and Actuators B: Chemical 2008, 129, 841.
Marcon, L., et al., Langmuir 2009, 26, 1075.
McMurray, R et al., Nature Materials 2011, 10, 637.
Pan, B., et al., J. Polym. Sci. Pol. Chem. 2004, 42, 1953.
Pasche et al., Langmuir, 2003, 19, 9216-9225
Priest., et al., Lab on a Chip 2010, 11, 541
Siow, et al., Plasma Processes and Polymers 2006, 392-418
Szunerits, S., and Boukherroub, R., Journal of Solid State Electrochemistry 2008, 12, 1205.
Wald, J.; Müller, C.; Wahl, M.; Hoth-Hannig, W.; Hannig, M.; Kopnarski, M.; Ziegler, C. physica status solidi (a) 2010, 207, 831-836.
Ward, A. J.; Short, R. D. Polymer 1993, 34, 4179-4185.
Ward, A. J.; Short, R. D. Polymer 1995, 36, 3439-3450.
Wickstrom, M., et al., The novel melphalan prodrug j1 inhibits neuroblastoma growth in vitro and in vivo Molecular Cancer Therapeutics, 2007. 6(9): p. 2409-2417.
Wu, P.; Castner, D. G.; Grainger, D. W. J. Biomater. Sci., Polym. Ed. 2008, 19, 725-753.
Zou et al., Journal of Membrane Science, 2011, 369, 420-428.

1. A method of patterning a surface of a substrate comprising:
(a) applying a coating to the surface to form a coated surface, and
(b) treating the coated surface with a patterned microplasma comprising a plurality of localised microplasma discharges such that localised regions of the coated surface are selectively exposed to the localised microplasma discharges to form exposed localised regions and unexposed regions that have not been substantially exposed to a microplasma discharge;
wherein the coating at the exposed localised regions is modified by the patterned microplasma and the coating at the unexposed regions is substantially unmodified to form a patterned surface on the substrate.

2. The method of claim 1 further comprising:
(c) treating the patterned surface with a binding agent that binds at the exposed localised regions.

3. The method of claim 2 wherein the coating at the unexposed regions inhibits binding of the binding agent at the unexposed regions.

4. The method of claim 2 wherein the coating comprises a biological material.

5. The method of claim 4 wherein the biological material is selected from the group consisting of: proteins, carbohydrates, fats, polynucleotides, biological fluids, fragments thereof, extracts thereof, and combinations thereof.

6. The method of claim 5 wherein the biological material is an albumin.

7. The method of claim 5 wherein the albumin is bovine serum albumin (BSA).

8. The method of claim 1 wherein the coating comprises a synthetic material.

9. The method of claim 8 wherein the synthetic material comprises one or more materials selected from a surfactant, a silane coupling agent, a hydrogel, a hydrocarbon, tetraethylene glycol dimethyl ether (tetraglyme), triethylene glycol dimethyl ether (triglyme), diethylene glycol dimethyl ether (diglyme), and a polymer selected from the group consisting of: glycol polymers, poly(ethylene glycol), ene polymers, octadiene polymers, polysaccharides, cellulose derivatives, agarose, alginic acid, poly N-isopropylacrylamide (PNIPAM), dextrans, phosphocholines, poly(hydroxethyl methacrylate), hyaluronic acid, pegylated SAMS, phosphorylcholine, poly(methyl-oxazoline) (PMOXA), graft copolymers of cationic polyelectrolyes and poly(ethylene glycol), graft copolymers of poly(L-lysine) and poly(ethylene glycol), graft copolymers of poly(ethylene imine) and poly(ethylene glycol), graft copolymers of cationic polyelectrolyes and dextran, graft copolymers of poly(L-lysine) and dextran, graft copolymers of poly(ethylene imine) and dextran, polyvinylpyrrolidone (PVP), polyvinyl alcohol, poly(hexamethylene disiloxane), poly(acrylic acid), polyacrylamide, fluoropolymers and poly(tetrafluoroethylene) (PTFE).

10. The method of claim 2 wherein the binding agent is selected from the group consisting of a tissue, a virus, a cell, an extra cellular component, a protein, a glycoprotein, a carbohydrate, a fat, a polynucleotide, a biological fluid, and fragments thereof.

11. The method of claim 10 wherein the binding agent comprises a cell.

12. The method of claim 11 wherein the cell is selected from the group consisting of: stem cells, including embryonic, mesenchymal, and induced pluripotent stem cells; mesoblasts, and mesenchymal progenitor cells.

13. The method of claim 12 wherein the surface is a cell expansion surface.

14. The method of claim 11 wherein the cell is selected from the group consisting of: neuroblastoma cells, lymphocyte B cells, and human epithelial carcinoma cells.

15. The method of claim 1 wherein the surface of the substrate is an open surface that is substantially planar.

16. The method of claim 1 wherein the patterned microplasma is patterned by a template that mediates selective exposure of the localised regions to the patterned microplasma, wherein the template does not contact the surface of the substrate.

17. The method of claim 1 wherein the surface of the substrate is an enclosed surface.

18. The method of claim 17 wherein the surface of the substrate is a microchannel.

19. The method of claim 1 wherein the microplasma is a helium microplasma.

20. A substrate comprising patterned surface produced by the method of claim 1.

21. A substrate comprising a patterned surface, wherein the patterned surface comprises a coating that has been modified at localised regions by selective exposure to patterned microplasma to form exposed localised regions and substantially unmodified unexposed regions, and wherein a target biological agent is optionally bound at the exposed localised regions.

22. (canceled)
